(19) United States
(12) Patent Application Publication
(10) Pub. No.: US 2023/0061503 A1
(43) Pub. Date: Mar. 2, 2023

(54) METHODS OF MINIMIZING NEUROTOXICITY ASSOCIATED WITH CHIMERIC ANTIGEN RECEPTOR (CAR) T CELL THERAPY

(71) Applicants: Janssen Biotech, Inc., Horsham, PA (US); Legend Biotech USA Inc., Piscataway, NJ (US)

(72) Inventors: Muhammad S. AKRAM, Monroe, NJ (US); Kevin C. De Braganca, Chappagua, NY (US); Jenna Goldberg, Livingston, NJ (US); Carolyn Chang Jackson, West Orange, NJ (US); Erin C. Lee, Yardley, PA (US); Nikoletta Lendvai, West Windsor, NJ (US); Maria Marquez de Mondelo, Belle Mead, NJ (US); Yunsi Olyslager, Kalmthout (BE); Jun Qiu, Kennett Square, PA (US); Enrique Zudaire Ubani, Spring House, PA (US); Jean Xu, Hillsborough, NJ (US)

(21) Appl. No.: 17/740,665

(22) Filed: May 10, 2022

Related U.S. Application Data
(60) Provisional application No. 63/186,872, filed on May 11, 2021.

(51) Int. Cl. A61K 35/17; A61P 35/00; A61K 31/573; A61K 45/06; G01N 33/68

(52) U.S. Cl. CPC A61K 35/17; A61P 35/00; A61K 31/573; A61K 45/06; G01N 33/6863; A61K 2039/505

(57) ABSTRACT

The present disclosure relates to methods of early identification and detection of neurotoxicity associated with the administration of chimeric antigen receptor (CAR) T cell therapy and mitigation strategies to reduce the occurrence and/or severity of treatment associated neurotoxicity.

### CROSS-REFERENCE TO RELATED APPLICATION

This application claims priority to U.S. Provisional Application Ser. No. 63/186,872, filed 11 May 2021, the entire contents of which is incorporated herein by reference in its entirety.

### FIELD

The present disclosure relates to methods of reducing neurotoxicity associated with chimeric antigen receptor (CAR) T cell therapy.

### BACKGROUND

Multiple myeloma (MM) is an incurable, malignant, plasma cell disorder that accounts for approximately 10% of hematological malignancies (Rodriguez-Abreu et al., “Epidemiology of Hematological Malignancies,” Ann. Oncol. 18 Suppl. 1:i3-i8 (2007) and Rajkumar et al., “Consensus Recommendations for the Uniform Reporting of Clinical Trials: Report of the International Myeloma Workshop Consensus Panel 1,” Blood 117(8):4691-4695 (2011)). Multiple myeloma is characterized by the proliferation of neoplastic clones of plasma cells differentiated from B-lymphocytes (B-cells). These neoplastic clones grow in the bone marrow, frequently invade the adjacent bone, disrupt both bone homeostasis and hematopoiesis, and cause multifocal destructive lesions throughout the skeleton that result in bone pain and fracture (Chung, C., “Role of Immunotherapy in Targeting the Bone Marrow Microenvironment in Multiple Myeloma: An Evolving Therapeutic Strategy,” Pharmacotherapy 37(1):129-143 (2017)).

Worldwide, there were an estimated 80,000 deaths due to MM (Ferlay et al., “Cancer Incidence and Mortality Patterns in Europe: Estimates for 40 Countries in 2012,” Eur. J. Cancer 49(6):1374-1403 (2013)). The estimated 5-year survival rate for patients with MM is approximately 54%. Despite multiple therapeutic options, the disease most often recurs and remains incurable. With each successive relapse, symptoms return, quality of life worsens, and the chance and duration of response typically decreases.

Standard treatment options for multiple myeloma include immunomodulatory imide drugs, proteasome inhibitors, anti-CD38 antibodies, and autologous stem cell transplant. However, because these approaches often fail or the disease becomes refractory, improved treatments are warranted.

Autologous chimeric antigen receptor (CAR)-T cell therapy is new form of cancer immunotherapy that involves engineering a patient's own T cells to identify and kill cancer cells within the patient. The use of a patient's own immune cells to eradicate cancer has shown to be a very promising approach in the treatment of leukemia and lymphoma, and is rapidly advancing to other cancers, such as multiple myeloma, that are in need of alternative therapies.

Unfortunately, CAR-T cell therapy can have side effects. Severe and potentially fatal neurotoxicity has been associated with CAR-T therapy targeting the CD19 antigen in leukemia and lymphoma. Neurotoxicity can occur concurrently with cytokine release syndrome (CRS) or after CRS resolution (see Yescarta® United States Product Insert (USPI)/Summary of Product Characteristics (SmPC); Kymriah® USPI/SmPC; Tecaratus® USPI/SmPC; Breyanzi® USPI; ABECMA® USPI). Immune effector cell-associated neurotoxicity syndrome (ICANS) has been well described in the literature; symptoms or signs can be progressive and may include aphasia, altered level of consciousness, impairment of cognitive skills, motor weakness, seizures, and cerebral edema (Lee et al., “ASTCT Consensus Grading for Cytokine Release Syndrome and Neurologic Toxicity Associated with Immune Effector Cells,” Biol. Blood Marrow Transplant 25(4):625-638 (2018); Neelapu et al., “Axicabtagene Ciloleucel CAR T-Cell Therapy in Refractory Large B-Cell Lymphoma,” N. Engl. J Med. 377:2531-2544 (2017); Santomasso et al., “Clinical and Biological Correlates of Neurotoxicity Associated with CAR T-Cell Therapy in Patients with B-Cell Acute Lymphoblastic Leukemia,” Cancer Discov. 8:958-971 (2018); and Schuster et al., “Tisagenlecleucel in Adult Relapsed or Refractory Diffuse Large B Cell Lymphoma,” N. Engl. J. Med. 380(1):45-56 (2019)). In the CD19 CAR-T experience, the incidence of ICANS is reported to be in the range of 35% to 87% (Kymriah® USPI/SmPC, Yescarta® USPI/SmPC, Breyanzi® USPI). In the BCMA CAR-T experience with idecabtagene vicleucel (also known as bb2121 and hereafter, referred to as ide-cel [ABECMA® USPI]), the overall incidence of neurotoxic effects ranges from 18% (Munshi et al., “Idecabtagene Vicleucel in Relapsed and Refractory Multiple Myeloma,” N. Engl. J. Med. 384:705-716 (2021) to 42% (Raje et al., “Anti-BCMA CAR T-Cell Therapy bb2121 in Relapsed or Refractory Multiple Myeloma,” N. Engl. J. Med. 380(18):1726-1737 (2019)). Specifically, the most frequently occurring CAR-T cell-associated neurotoxicities include encephalopathy (20%), tremor (9%), aphasia (7%), and delirium (6%) with events of Grade 3 parkinsonism and Grade 4 cerebral edema also reported. The median time of onset for CAR-T cell-associated neurotoxicity after ide-cel treatment was 2 days (range: 1 day to 42 days), and the median duration of the neurotoxic events was 6 days (range: 1 to 578 days) (ABECMA® USPI).

Ciltacabtagene autoleucel (cilta-cel) is a genetically modified autologous T-lymphocyte (T-cell) immunotherapy for refractory multiple myeloma that binds to B-cell maturation antigen (BCMA). In clinical studies with cilta-cel, CAR-T neurotoxicity has been observed and categorized as ICANS as well as Other Neurotoxicities determined to be related to CAR-T therapy and occurring after recovery of CRS and/or ICANS. An understanding of the factors contributing to CAR-T cell neurotoxicity is needed, as are mitigation strategies to reduce and/or prevent CAR-T cell neurotoxicity all together to improve treatment outcomes.

The present disclosure is aimed at overcoming this and other deficiencies in the art.

### SUMMARY

A first aspect of the present disclosure is directed to a method of reducing neurotoxicity associated with chimeric antigen receptor (CAR) T cell therapy. This method involves administering CAR-T cell therapy to a subject and determining one or more of (i) the subject's tumor burden prior to said administering, (ii) the subject's IL-6 levels at the time of said administering, (iii) CAR-T cell expansion in said subject after said administering, (iv) CAR-T cell persistence in peripheral blood of said subject after said administering, (v) development of grade ≥2 cytokine release syndrome (CRS) in said subject after said administering, (vi) development of immune effector cell associated neurotoxicity syndrome (ICANS) in said subject after said administering, (vii) peak peripheral blood levels of IL-6 in said subject following said administering, (viii) peak peripheral blood levels of INF-γ in said subject after said administering; and (iv) lymphocyte counts in said subject after said administering. The method further involves administering a mitigating therapeutic to said subject, based on said determining, to reduce neurotoxicity associated with CAR-T cell therapy.

Another aspect of the present disclosure is directed to a method for treating multiple myeloma in a subject with chimeric antigen receptor (CAR) T cell therapy while reducing neurotoxicity associated with the therapy. This method involves administering CAR-T cell therapy to a subject having multiple myeloma, where the subject has a tumor burden characterized by bone marrow plasmacytosis of <80%, serum M Protein levels of <5 g/dL, and serum free light chain levels of <5000 mg/L.

Another aspect of the present disclosure is directed to a method for treating multiple myeloma in a subject with chimeric antigen receptor (CAR) T cell therapy while reducing neurotoxicity associated with said therapy. This method involves administering CAR-T cell therapy to a subject having multiple myeloma and having an IL-6 serum level that is within the normal reference range, for example 0-2 pg/mL.

A further aspect of the present disclosure is directed to a method of reducing neurotoxicity in a subject receiving chimeric antigen receptor (CAR) T cell therapy for the treatment of multiple myeloma. This method involves administering to a subject having received CAR-T cell therapy and having symptoms of CAR-T cell therapy associated cytokine release syndrome (CRS) or having symptoms of immune effector cell associated neurotoxicity syndrome (ICANS), an anti-inflammatory agent in an amount effective to reduce neurotoxicity in said subject.

Yet a further aspect of the present disclosure is directed to a method of reducing neurotoxicity in a subject receiving chimeric antigen receptor (CAR) T cell therapy for the treatment of multiple myeloma. This method involves administering to a subject having received CAR-T cell therapy and having a CAR-T cell maximum plasma concentration (Cₘₐₓ) of >1,000 cells/μL and/or a persisting CAR-T cell concentration in peripheral blood of >300 cells/μL after CAR-T cell administration (e.g., at 45-100 days after CAR-T cell administration), a chemotherapeutic to reduce CAR-T cell therapy associated neurotoxicity.

Another aspect of the present disclosure is directed to a method of reducing neurotoxicity in a subject receiving chimeric antigen receptor (CAR) T cell therapy for the treatment of multiple myeloma. This method involves administering, to the subject having received CAR-T cell therapy and having a peak peripheral blood IL-6 level that is above the upper limit of normal peripheral blood IL-6 levels after CAR-T cell administration, an IL-6 inhibitor to reduce CAR-T cell therapy associated neurotoxicity.

Another aspect of the present disclosure is directed to a method of reducing neurotoxicity in a subject receiving chimeric antigen receptor (CAR) T cell therapy for the treatment of multiple myeloma. This method involves administering, to the subject having received CAR-T cell therapy and having a peak peripheral blood INF-γ level above the upper limit of normal peripheral blood INF-γ levels after CAR-T cell administration, an INF-γ inhibitor to reduce CAR-T cell therapy associated neurotoxicity.

Cilta-cel is a CAR-T-cell therapy with two BCMA-targeting, single-domain antibodies designed to bind and destroy malignant cells. In Phase 1b and Phase 2 clinical studies with cilta-cel, CAR-T neurotoxicity was observed and categorized as ICANS as well as Other Neurotoxicities related to CAR-T therapy and occurring after recovery of CRS and/or ICANS. As described herein, based on those studies, several factors that correlate with the development of cilta-cel CAR-T cell associated Other Neurotoxicity were identified, and based on those factors, mitigation and management strategies have been developed. These strategies were employed in Phase 2 and Phase 3 studies assessing the efficacy of cilta-cel in patients with multiple myeloma. The results show that with the inclusion of mitigation and management strategies, neurotoxic events are generally manageable in patients following treatment with cilta-cel. In fact, only 1 out of 100+ patients receiving cilta-cel experienced neurotoxic adverse events. These results indicate that early detection and management of neurologic adverse events leads to better CAR-T cell treatment outcomes.

### BRIEF DESCRIPTION OF THE DRAWINGS

FIG. 1 is a schematic illustration showing an overview of chimeric antigen receptor T-cell (CAR-T) neurotoxicities in the all treated analysis set of clinical study 68284528MMY2001. AE=adverse event; ICANS=immune effector cell-associated neurotoxicity syndrome; SOC=system organ class; TEAE=treatment emergent adverse event.

FIG. 2 is a table showing the pharmacokinetic data of subjects with peripheral blood CAR-T cell Cₘₐₓ of >1,000 cells/μL and CAR-T Cells >300 cells/μL at Day 56. CAR-T=chimeric antigen receptor T-cell; Cₘₐₓ=maximum plasma concentration; ID=identification; ND=not determined; TEAE=treatment-emergent adverse event. Red circle=subjects with movement or neurocognitive TEAEs. Note: Subject L28US10021005 showed the subject's CAR+CD3+ T cells at maximum level of 1,750 cells/μL at Day 14. The concentration was at 362 cells/μL at the time of onset of symptoms (Day 28). After Day 56, they rapidly declined to 7 cells/μL.

FIG. 3 is a plot showing baseline IL-6 in the all treated analysis set of clinical study 68284528MMY2001. TEAE=treatment-emergent adverse event. Note: One subject having movement and neurocognitive TEAE did not have baseline IL-6 levels reported.

FIG. 4 is a plot showing lymphocytes (×10⁹/L) over time in the all treated analysis set for clinical study 68284528MMY2001. TEAE=treatment-emergent adverse event.

FIG. 5 is a plot showing peak IL-6 levels (Cₘₐₓ) for the all treated analysis set in clinical study 68284528MMY2001. Cₘₐₓ=maximum concentration; M&NC=movement and neurocognitive TEAEs; NTX=neurotoxicity; TEAE=treatment-emergent adverse event.

FIG. 6 is a plot showing interferon γ peak levels (Cₘₐₓ) in the all treated analysis set for clinical study 68284528MMY2001. Cₘₐₓ=maximum concentration; M&NC=movement and neurocognitive TEAEs; NTX=neurotoxicity; TEAE=treatment-emergent adverse event.

FIG. 7 shows the frequency distribution of CD4 and CD8 T cells of naïve/stem cell memory (Tn/Tscm), central memory (Tcm), effector memory (Tem) and effector memory re-expressing CD45RA (Temra) phenotype. TEAE=treatment-emergent adverse event; NTX=neurotoxicity; ICANS=immune effector cell-associated neurotoxicity event.

FIGS. 8A-8B shows a comparison of the total number of CAR-positive viable T-cells administered (with or without body weight normalization) (FIG. 8A), predicted CAR transgene Cₘₐₓ, AUC_{0-28d}, and Tₘₐₓ (FIG. 8B) between subjects with and without other neurotoxicities in clinical study 68284528MMY2001. AUC0-28 d=area under the CAR transgene systemic level-time curve from the first dose to Day 28; CAR=chimeric antigen receptor; Cₘₐₓ=maximum CAR transgene systemic level; gDNA=genomic DNA; ICANS=immune effector cell-associated neurotoxicity syndrome; TEAE=treatment-emergent adverse event; Tₘₐₓ=time of maximum CAR transgene. Other neurotoxicities refer to other events of CAR-T neurotoxicity not defined as ICANS. Red dots represent subjects with movement and neurocognitive TEAEs.

FIG. 9 shows a comparison of predicted Cₘₐₓ, AUC_{0-28d} and Tₘₐₓ between subjects without and with movement and neurocognitive TEAEs. AUC_{0-28d}=area under the CAR transgene systemic level-time curve from the first dose to Day 28; CAR=chimeric antigen receptor; Cₘₐₓ=maximum CAR transgene systemic level; gDNA=genomic DNA; ICANS=immune effector cell-associated neurotoxicity syndrome; TEAE=treatment-emergent adverse event; Tₘₐₓ=time of maximum CAR transgene.

FIG. 10 is a table showing the listing of tumor burden and bridging therapy in subjects with movement and neurocognitive TAEAs in the all treated analysis set for clinical study 68284528MMY2001.

FIG. 11 is a forest plot of potential factors associated with movement and neurocognitive TEAE in the all treated analysis set for clinical study 68284528MMY2001. ALC=absolute lymphocyte count; CBC=complete blood count; CRS=cytokine release syndrome; ICANS=Immune Effector Cell-Associated Neurotoxicity Syndrome; OR=odds ratio.

FIG. 12 is a table listing the movement and neurocognitive treatment-emergent adverse events in the all treated analysis set for clinical study 68284528MMY2001.

FIG. 13 is a plot showing baseline IL-10 in the all treated analysis set for clinical study 68284528MMY2001. TEAE=treatment-emergent adverse event.

FIG. 14 is a plot showing baseline interferon gamma in the all treated analysis set for clinical study 68284528MMY2001. TEAE=treatment-emergent adverse event.

FIG. 15 is a plot showing baseline interleukin 2 receptor subunit alpha in the all treated analysis set for clinical study 68284528MMY2001. TEAE=treatment-emergent adverse event.

### DETAILED DESCRIPTION

The present disclosure relates to methods of early identification and detection of neurotoxicity associated with the administration of chimeric antigen receptor (CAR) T cell therapy and mitigation strategies to reduce the occurrence and/or severity of treatment associated neurotoxicity.

CAR-T neurotoxicity is categorized as (i) immune effector cell-associated neurotoxicity syndrome (ICANS) and (ii) Other Neurotoxicity (i.e., non-ICANS). Other Neurotoxicity is determined by a medical professional to be related to CAR-T therapy and occurring after recovery from cytokine release syndrome and/or ICANS. In any embodiment, the methods described herein are directed to methods of detecting and reducing the non-ICANS neurotoxicity events associated with CAR-T cell therapy, which are collectively referred to herein as “CAR-T cell associated neurotoxicity”, “neurotoxicity associated with CAR-T cell therapy”, and “Other Neurotoxicity”.

In accordance with the methods described herein, adverse neurotoxicity events associated with the administration of CAR-T cell therapy are characterized as movement and motor dysfunction treatment-emergent adverse event (TEAE), cognitive impairment TEAE, personality change TEAE, or any combination thereof.

In any embodiment herein, movement and motor dysfunction TEAEs that are characteristic of CAR-T cell associated neurotoxicity (i.e., non-ICANS) include, without limitation, ataxia, balance disorder, bradykinesia, cogwheel rigidity, dysgraphia, dyskinesia, dysmetria, essential tremor, gait disturbance, hand-eye coordination impaired, micrographia, motor dysfunction, myoclonus, parkinsonism, posture abnormal, resting tremor, stereotypy, and tremor. Thus, the methods described herein reduce, minimize, inhibit the onset of, or prevent any one or more of the above noted movement and motor dysfunction adverse events associated with CAR-T cell therapy.

In any embodiment herein, cognitive impairment TEAEs that are characteristic of CAR-T cell associated neurotoxicity (i.e., non-ICANS) include, without limitation, amnesia, apraxia, bradyphrenia, cognitive disorder, confusional state, depressed level of consciousness, disturbance in attention, encephalopathy, incoherent, leukoencephalopathy, loss of consciousness, memory impairment, mental impairment, mental status changes, noninfective encephalitis, and psychomotor retardation. Thus, the methods described herein reduce, minimize, inhibit the onset of, or prevent any one or more of the above noted cognitive impairment adverse events associated with CAR-T cell therapy.

In any embodiment herein, a personality change TEAEs that are characteristic of CAR-T cell associated neurotoxicity (i.e., non-ICANS) include, without limitation, flat affect, personality change, or reduced facial expression. Thus, the methods described herein reduce, minimize, inhibit the onset of, or prevent any one or more of the above noted personality adverse events associated with CAR-T cell therapy.

Accordingly, a first aspect of the present disclosure is directed to a method of reducing neurotoxicity associated with chimeric antigen receptor (CAR) T cell therapy. This method involves administering CAR-T cell therapy to a subject and determining one or more factors associated with the development of CAR-T cell neurotoxicity. These factors include (i) the subject's tumor burden prior to said administering, (ii) the subject's IL-6 levels at the time of said administering, (iii) CAR-T cell expansion in said subject after said administering, (iv) CAR-T cell persistence in peripheral blood of said subject after said administering, (v) development of grade ≥2 cytokine release syndrome (CRS) in said subject after said administering, (vi) development of immune effector cell associated neurotoxicity syndrome (ICANS) in said subject after said administering, (vii) peak peripheral blood levels of IL-6 in said subject following said administering, (viii) peak peripheral blood levels of INF-γ in said subject after said administering; and (iv) lymphocyte counts in said subject after said administering.

CAR-T cell therapy is utilized to treat various conditions including multiple myeloma, various B-cell lymphomas, e.g., mantle cell lymphoma, follicular lymphoma, high grade B-cell lymphoma, aggressive B-cell lymphoma, large B-cell lymphoma, primary mediastinal large B-cell lymphoma, diffuse large B-cell lymphoma, and B-cell precursor acute lymphoblastic leukemia (ALL). Neurotoxicity is a side-effect associated with CAR-T cell administration in the above conditions, and thus the methods described herein are applicable to reducing neurotoxicity, in particular the non-ICANS neurotoxicity, in a subject receiving CAR-T cell therapy for any of the above noted conditions. In any embodiment, the subject treated in accordance with the methods described herein is receiving CAR-T cell therapy for the treatment of multiple myeloma.

In any embodiment, the subject treated in accordance with the methods described herein has multiple myeloma and is receiving CAR-T cell therapy where the CAR targets B-cell maturation agent (BCMA). In any embodiment, the subject has multiple myeloma and is receiving idecabtagene vicleucel or ciltacabtagene autoleucel (cilta-cel). In any embodiment, the subject has multiple myeloma and is receiving ciltacabtagene autoleucel (cilta-cel) as fully described in WO2017/025038 to Fan et al. and WO2018/028647 to Fan et al., which are hereby incorporated by reference in their entirety. Suitable methods of administering CAR-T cell therapy for the treatment of a various lymphoid neoplasms are known in the art (see e.g., Cerrano et al., “The Advent of CAR T-Cell Therapy for Lymphoproliferative Neoplasms: Integrating Research Into Clinical Practice,” Front. Immunol. 11(888) (2020), which is hereby incorporated by reference in its entirety). Methods of administering BCMA CAR-T cell therapy for the treatment of multiple myeloma are also known in the art (see e.g., WO2017/025038 to Fan et al. and WO2018/028647 to Fan et al., which are hereby incorporated by reference in their entirety), and are described herein.

In any embodiment, methods of reducing neurotoxicity associated with CAR-T cell therapy can involve determining at least two of the above noted factors, determining at least three of the factors, determining at least four of the factors, determining at least five of the factors, determining at least six of the factors, determining at least seven of the factors, or determining at least eight of the factors. In some embodiments, the method of reducing neurotoxicity associated with CAR-T cell therapy involves determining all nine of the above noted factors.

In accordance with this aspect of the disclosure, depending on the outcome of the analysis of the one or more factors above, one or more mitigating strategies as described herein are employed to reduce, prevent, or minimize neurotoxicity associated with CAR-T cell therapy. In some embodiments, multiple mitigating strategies as described herein are employed to reduce neurotoxicity associated with CAR-T cell therapy. In some embodiments, the mitigating strategy involves the administration of a therapeutic agent. In some embodiments, the mitigating strategy involves further observation and monitoring of the subject to detect early signs of neurotoxicity.

For the purposes of this disclosure, “reducing” neurotoxicity associated with CAR-T cell therapy includes, but is not limited to, alleviation of movement, cognitive, and/or personality adverse events that arise after CAR-T cell treatment; diminishment of the extent of any or all movement, cognitive, and/or personality adverse events associated with treatment; stabilization (i.e., not worsening) of any or all movement, cognitive, and/or personality adverse events; delay in onset or slowing of the progression of any or all movement, cognitive, and/or personality adverse events; or amelioration of any or all movement, cognitive, and personality adverse events. In some embodiments, the methods of reducing neurotoxicity as described herein are preemptive in nature, i.e., CAR-T cell associated neurotoxicity is prevented. Prevention may involve complete protection from CAR-T cell associated neurotoxicity events, or may involve prevention of CAR-T cell associated neurotoxicity progression. For example, prevention may not mean complete foreclosure of any neurotoxicity event associated with CAR-T cell treatment at any level, but instead may mean prevention of the symptoms to a clinically significant or detectable level. Prevention of CAR-T cell associated neurotoxicity may also mean prevention of the progression of the neurotoxicity to a later stage as compared to the progression experienced by a subject not administered a mitigating treatment as described herein.

In any embodiment, the method of reducing neurotoxicity associated with CAR-T cell therapy involves determining the subject's tumor burden prior to administering the CAR-T cell therapy. As described herein, a high tumor burden in the subject at the time the CAR-T cell therapy is administered is associated with the development of CAR-T cell associated neurotoxicity. The subject's tumor burden can be determined using methods standard in the art for a particular tumor. For example, when the subject has multiple myeloma, the subject's tumor burden can be determined by measuring the subject's level of plasmacytosis in the bone marrow, serum level of M protein, and/or serum level free light chains.

Plasmacytosis in the bone marrow, i.e., the percentage of bone marrow cells that are plasma cells, can be determined in a bone marrow biopsy or aspirate. The number of plasma cells can be determined by immunohistochemical or flow cytometry techniques using a combination of identifying antibodies, including, without limitation, antibodies to CD138 or VS38c, Bcl-2, CD79a, and CD20. Plasma cells normally account for ˜2%-3% of bone marrow cells. In accordance with the present disclosure, a finding that plasma cells account for ≥30%, ≥40%, ≥50%, ≥60%, ≥70%, or ≥80% of the subject's bone marrow cells is indicative that the subject has a high tumor burden. In any embodiment, a finding that plasma cells account for ≥80% of the subject's bone marrow cells is indicative that the subject has a high tumor burden.

M proteins, also referred to myeloma proteins, monoclonal immunoglobulin, M spike or paraprotein, are bone marrow derived antibodies released from myeloma cells that can be detected and quantified using serum or urine electrophoresis, immunofixation electrophoresis of blood or urine, or quantitative immunoglobulin testing. A finding that a subject's serum M protein levels are ≥2 g/dL, ≥3 g/dL, ≥4 g/dL, ≥5 g/dL is indicative that the subject has a high tumor burden. In any embodiment, a finding that the subject's serum M protein levels are ≥5 g/dL is indicative that the subject has a high tumor burden.

In some instances, the myeloma cells do not produce the whole M protein (i.e., a complete immunoglobulin) and instead release just the light chains, i.e., free immunoglobulin kappa (κ) and lambda (λ) free light chains. These free light chains, also referred to as Bence Jones proteins, can be detected in the blood or urine using light chain specific antibodies (see e.g., Bradwell et al., Highly Sensitive, Automated Immunoassay for Immunoglobulin Free Light Chains in Serum and Urine,” Clin. Chem 47(4):673-80 (2001), which is hereby incorporated by reference in its entirety). A normal range for κ and λ chain concentration in the serum of healthy individuals is ˜3.3-19.4 mg/L and ˜5.7-26.6 mg/L for the κ and λ chains, respectively, and a ratio of kappa/lambda of 0.26 to 1.65. A finding that a subject has serum free light chain levels of >100 mg/L, >500 mg/L, >1000 mg/L, >2000 mg/L, >3000 mg/L, >4000 mg/L, ≥5000 mg/L is indicative that the subject has a high tumor burden. In any embodiment, a finding that a subject has serum free light chain levels of ≥5000 mg/L is indicative that the subject has a high tumor burden.

If it is determined that a subject has a high tumor burden at the time the CAR-T cell therapy is going to be administered, the risk of developing neurotoxicity associated with CAR-T cell therapy can be mitigated by administering a bridging therapy prior to administering the CAR-T cell therapy to reduce the subject's tumor burden. A “bridging therapy” is any therapy suitable for reducing tumor burden between leukapheresis, i.e., when the subject's T cells are collected, and conditioning, i.e., when the subject is given conditioning chemotherapy in anticipation of receiving the CAR-T cell therapy. In some embodiments, the bridging therapy is administered in an amount that is effective to reduce the subject's tumor burden to achieve a tumor burden characterized by bone marrow plasmacytosis of <80%, serum M Protein levels of <5 g/dL, and serum free light chain levels of <5000 mg/L prior to administering the CAR-T cell therapy. In some embodiments, the bridging therapy is administered in an amount that is effective to reduce the subject's tumor burden to achieve a bone marrow plasmacytosis of <50%, serum M protein levels of <3 g/dL, and serum free light chain levels of <3000 mg/L prior to administering the CAR-T cell therapy.

Suitable bridging therapies include, without limitation, a chemotherapeutic, an immunomodulatory agent, a proteasome inhibitor, or any combination thereof.

In any embodiment, the bridging therapy is chemotherapy. Suitable chemotherapeutics include alkylating agents, such as cyclophosphamide (Cytoxan), melphalan, melfulfen (Pepaxto®), and bendamustine (Treanda®), and topoisomerase inhibitors, such as etoposide (VP-16) and doxorubicin (Adriamycin, Doxil).

In any embodiment, the bridging therapy is a proteasome inhibitor. Suitable proteasome inhibitors to administer as a bridging therapy include, without limitation, bortezomib (Velcade®), carfilzomib (Kyprolis®), and ixazomib (Ninlaro®). In any embodiment, the aforementioned proteasome inhibitors can be administered in combination with dexamethasone, dexamethasone and lenalidomide, or dexamethasone and cyclophosphamide.

In any embodiment, the bridging therapy is an immunomodulatory agent. Suitable immunomodulatory agents for a subject having multiple myeloma include, without limitation, CD38 inhibitors and SLAMF7 inhibitors. Suitable CD38 inhibitors include anti-CD38 monoclonal antibodies such as daratumamab (Darzalex®) and isatuximab (Sarclisa®). A suitable SLAMF7 inhibitor includes the monoclonal anti-SLAMF7 antibody, elotuzumab (Empliciti®). Other suitable immunomodulatory agents include, without limitation, lenalidomide (Revlimid®), pomalidomide (Pomalyst®), thalidomide, and combinations thereof. In any embodiment, the aforementioned immunomodulatory agents can be administered in combination with dexamethasone.

Other suitable bridging therapies include, without limitation, histone deacetylase (HDAC) inhibitors, such as panobinostat (Farydak®); nuclear export inhibitors, such as selinexor (Xpovio®); and antibody drug conjugates, such as belantamab mafodotin-blmf (Blenrep).

In some embodiments, the bridging therapy comprises a combination of any of the aforementioned chemotherapeutic agents, proteasome inhibitors, or immunomodulatory agent. Suitable combination therapies include, without limitation, lenalidomide (or pomalidomide or thalidomide) and dexamethasone; carfilzomib (or ixazomib or bortezomib), lenalidomide, and dexamethasone; bortezomib (or carfilzomib), cyclophosphamide, and dexamethasone; elotuzumab, lenalidomide, and dexamethasone; daratumumab, lenalidomide, and dexamethasone; isatuximab, lenalidomide, and dexamethasone; bortezomib, liposomal doxorubicin, and dexamethasone; panobinostat, bortezomib, and dexamethasone; elotuzumab, bortezomib, and dexamethasone; melphalan and prednisone (MP), with or without thalidomide or bortezomib; vincristine, doxorubicin (Adriamycin), and dexamethasone (called VAD); dexamethasone, cyclophosphamide, etoposide, and cisplatin (called DCEP); dexamethasone, thalidomide, cisplatin, doxorubicin, cyclophosphamide, and etoposide (called DT-PACE), with or without bortezomib; and selinexor, bortezomib, and dexamethasone

In any embodiment, the method of reducing neurotoxicity associated with CAR-T cell therapy involves determining the subject's IL-6 blood levels at the time of administering the CAR-T cell therapy. If the subject's IL-6 blood levels are above the upper limit of normal, then an IL-6 inhibitor is administered as a mitigating agent to reduce the IL-6 levels prior to administering the CAR-T therapy. Suitable IL-6 inhibitors include, without limitation tocilizumab (Actemra®), sarliumab (Kevzara®) siltuximab (Sylvant®), and clazakizumab (Atal and Fatima, “IL-6 Inhibitors in the Treatment of Serious COVID-19: A Promising Therapy,” PharmaceuticalMedicine 34:223-231 (2020), which is hereby incorporated by reference in its entirety). The reference range of normal of IL-6 is 0-2 pg/ml (see e.g., Wang et al., IL-6 Signaling in Peripheral Blood T Cells Predicts Clinical Outcome in Breast Cancer,” Cancer Research 77(5): 1119-1126 (2016), which is hereby incorporated by reference in its entirety). Accordingly, if the subject's IL-6 blood levels are >2 pg/mL, then tocilizumab or a similarly effective IL-6 inhibitor is administered to said subject to lower the IL-6 level to within the 0-2 pg/mL reference range prior to administering the CAR-T cell therapy.

In any embodiment, the method of reducing neurotoxicity associated with CAR-T cell therapy involves determining the subject's peak peripheral blood levels of IL-6 after the CAR-T cell therapy is administered. If the subject's IL-6 blood levels are above the upper limit of normal, then an IL-6 inhibitor, such as tocilizumab, sarliumab, siltuximab, or clazakizumab, is administered as a mitigating agent to reduce the IL-6 levels is. As noted above, the reference range of normal blood IL-6 levels is 0-2 pg/ml. Accordingly, if the subject's peak peripheral IL-6 blood levels are >2 pg/mL, then tocilizumab or a similarly effective IL-6 inhibitor is administered to the subject in an amount effective to lower the IL-6 level to within the 0-2 pg/mL reference range.

In any embodiment, the method of reducing neurotoxicity associated with CAR-T cell therapy involves determining the subject's peak peripheral blood levels of INF-γ after the CAR-T cell therapy is administered. If the subject's INF-γ blood levels are above the upper limit of normal, then an INF-γ inhibitor is administered as a mitigating agent to reduce the INF-γ levels to an acceptable level. Suitable INF-γ inhibitors, such the monoclonal IFN-7 antibody, emapalumab (Gamifant®) (Vallurupalli and Berliner, “Emapalumab for the Treatment of Relapsed/Refractory Hemophagocytic Lymphohistiocytosis,” Blood 134(21):1783-1786 (2019), which is hereby incorporated by reference in its entirety), that are known in the art are suitable for use in accordance with the methods described herein. Normal blood levels of INF-γ are about <2.0 pg/mL. Accordingly, if the subject's peak peripheral INF-γ blood levels are >2 pg/mL, then emapalumab or a similarly effective INF-γ inhibitor is administered to said subject to lower the INF-γ level to the <2 pg/mL reference range.

In any embodiment, the method of reducing neurotoxicity associated with CAR-T cell therapy involves determining the subject's lymphocyte counts after the CAR-T cell therapy is administered. If the subject's lymphocyte counts are above the upper limit of normal, at about 2 weeks, at about 3 weeks, at about 4 weeks, at about 5 weeks, at about 6 weeks, at about 7 weeks, at about 8 weeks, at about 9 weeks, or at about 10 weeks after the CAR-T therapy is administered, then an anti-inflammatory agent is administered as a mitigating agent to reduce lymphocyte counts. Suitable anti-inflammatory agents include, without limitation, IL-6 inhibitors, IL-1 receptor antagonists (e.g., anakinra), tyrosine kinase inhibitors (e.g., dasatinib), steroids, and methotrexate. Normal lymphocyte counts are in the range of 0.8-3.0×10⁹/L. Accordingly, if the subject's lymphocyte counts are above 3.0×10⁹/L, then an anti-inflammatory agent is administered to said subject to lower the lymphocyte counts until they are near or within the 0.8-3.0×10⁹/L reference range.

In any embodiment, the method of reducing neurotoxicity associated with CAR-T cell therapy involves determining CAR-T cell expansion and persistence in the subject after the CAR-T cell therapy is administered. If the subject's CAR-T cell expansion is high, which is defined as a CAR-T cell maximum plasma concentration (Cmax) of >1,000 cells/μL, a mitigating therapeutic is administered to reduce the number of CAR-T cells in the subject. Similarly, if CAR-T cell persistence is high, which is defined as a peripheral blood CAR-T cell level of >300 cells/μL at about 45-100 days after treatment, a mitigating therapeutic is administered to reduce the number of CAR-T cells persisting in the subject. Suitable mitigating therapeutics to reduce the number of CAR-T cells in the subject include, without limitation, chemotherapeutic and anti-inflammatory agents. Suitable chemotherapeutic agents are described supra and include alkylating agents and topoisomerase inhibitors. Suitable anti-inflammatory agents include, without limitation, IL-6 inhibitors, IL-1 receptor antagonists (e.g., anakinra), tyrosine kinase inhibitors (e.g., dasatinib), steroids, and methotrexate. If the subject's CAR-T cell expansion is high, the mitigating agent is administered in an amount and for a duration effective to reduce the plasma concentration of CAR-T cells to <1,000 cells/μL. If the subject's CAR-T cell peripheral persistence is high (e.g., at about 45-100 days following treatment), the mitigating agent is administered in an amount and for a duration effective to reduce number of peripheral blood CAR-T cells to <300 cells/μL.

In any embodiment, the method of reducing neurotoxicity associated with CAR-T cell therapy involves monitoring the subject for the development of cytokine release syndrome (CRS). As used herein, the term “cytokine release syndrome” or “CRS” refers to a supraphysiologic response following any immune therapy that results in the activation or engagement of endogenous or infused T cells and/or other immune effector cells (see e.g., Lee et al., “ASTCT Consensus Grading for Cytokine Release Syndrome and Neurologic Toxicity Associated with Immune Effector Cells,” Biol. Blood Marrow Transplant. 25(4):625-638 (2019), which is hereby incorporated by reference in its entirety). As described herein prior or concurrent CRS correlates with the development of CAR-T cell associated neurotoxicity. In particular, a subject that develops grade 2 or higher CRS is prone to developing CAR-T cell therapy associated neurotoxicity and should be treated aggressively with an anti-inflammatory agent to resolve the CRS. Suitable therapeutics for the treatment of CRS are disclosed infra.

Symptoms of CRS include, without limitation, fever, fatigue, myalgias, arthralgias, headache, nausea/vomiting, diarrhea, skin rash, tachypnea, hypoxia, pulmonary edema, elevated D-dimer, hypofibrinogenemia, renal dysfunction (e.g., azotemia), hepatic dysfunction (e.g., transaminitis and/or hyperbilirubinemia, cardiovascular dysfunction (e.g., tachycardia, hypotension, capillary leak, widened pulse pressure, modulated cardiac output). Grading of the CRS can be carried out using any of the known and acceptable grading scales as described in Riegler et al., “Current Approaches in the Grading and Management of Cytokine Release Syndrome after Chimeric Antigen Receptor T-cell Therapy,” Ther. Clin. Risk Manag. 15:323-335 (2019), which is hereby incorporated by reference in its entirety.

In any embodiment, the method of reducing neurotoxicity associated with CAR-T cell therapy involves monitoring the subject for the development of immune effector cell associated neurotoxicity syndrome (ICANS) after the CAR-T cell therapy is administered. As used herein, the term “immune effector cell-associated neurotoxicity syndrome” or “ICANS” refers to a disorder characterized by a pathologic process involving the central nervous system following any immune therapy that results in the activation or engagement of endogenous or infused T cells and/or other immune effector cells (see e.g., Lee et al., “ASTCT Consensus Grading for Cytokine Release Syndrome and Neurologic Toxicity Associated with Immune Effector Cells,” Biol. Blood Marrow Transplant. 25(4):625-638 (2019), which is hereby incorporated by reference in its entirety). As described herein prior or concurrent ICANS correlates with the development of CAR-T cell therapy associated neurotoxicity. In particular, a subject that develops any level of ICANS is prone to developing CAR-T cell therapy associated neurotoxicity and should be treated aggressively with an anti-inflammatory agent to resolve the ICANS. Suitable therapeutics for the treatment of ICANS are disclosed infra.

Symptoms of ICANS include, without limitation, delirium, encephalopathy, aphasia (expressive progressing to global aphasia), lethargy, difficulty concentrating, agitation, tremor, seizures, dysgraphia, mild difficulty with expressive speech, apraxia, and cerebral edema. Grading of ICANS can be carried out using any of the known and acceptable grading scales as described in Lee et al., “ASTCT Consensus Grading for Cytokine Release Syndrome and Neurologic Toxicity Associated with Immune Effector Cells,” Biol. Blood Marrow Transplant 25:625-638 (2019), which is hereby incorporated by reference in its entirety. In any embodiment, ICANS is graded according in accordance with the Immune effector Cell-associated Encephalopathy (ICE) Assessment Tool (ICE-Tool) as described herein and Lee et al. Biol. Blood Marrow Transplant 25:625-638 (2019), which is hereby incorporated by reference in its entirety.

Suitable methods and therapeutics for treating CRS and ICANS that are known in the art (see e.g., Riegler et al., “Current Approaches in the Grading and Management of Cytokine Release Syndrome after Chimeric Antigen Receptor T-cell Therapy,” Ther. Clin. Risk Manag. 15:323-335 (2019), which is hereby incorporated by reference in its entirety) are suitable for use in accordance with the methods described herein. The primary therapeutic agents administered to alleviate CRS are anti-inflammatory agents including, without limitation, IL-6 inhibitors (e.g., tocilizumab, sarliumab, siltuximab, or clazakizumab), IL-1 inhibitors (e.g., anakinra), Janus kinase 1/2 inhibitors (e.g., ruxolitinib) and corticosteroids, such as dexamethasone, methylprednisolone, hydrocortisone. In some embodiments, combinations of the aforementioned therapeutics, e.g., a combination of an IL-6 inhibitor and steroid, are administered to the subject to aggressively treat and resolve the CRS. Other suitable therapeutics for the treatment of CRS include anti-thymocyte globulin and cyclophosphamide. Treatment for ICANS primarily involves administration of corticosteroids (dexamethasone, methylprednisolone, hydrocortisone), but can also include anti-inflammatory agents (i.e., IL-6 inhibitor) if concurrent CRS is present.

The methods disclosed herein for reducing neurotoxicity associated with CAR-T cell therapy involve determining one or more of the factors associated with the development of CAR-T cell neurotoxicity as described above. In addition, however, the methods further involve monitoring the subject for symptoms of agraphia, micrographia, dysgraphia, or any combination thereof after administering the CAR-T cell therapy. This monitoring provides a means for early detection and identification of CAR-T cell therapy associated neurotoxicity. Symptoms of agraphia (i.e., the loss in ability to write), micrographia (i.e., a disorder featuring abnormally small, cramped handwriting or the progression to progressively smaller handwriting), and dysgraphia (i.e., characterized by difficulty or inconsistency in letter and word spacing, poor spelling, unfinished words, missing words or letters), or any combination thereof can be monitored by providing the subject periodic handwriting assessments. The handwriting assessment is administered prior to the administration of CAR-T cell therapy and periodically after the CAR-T cell therapy is administered to assess and detect any changes as early as possible. Ongoing assessments can be carried out for 1 month, 2 months, 3 months, 4 months, 5 months, 6 months, 7 months, 8 months, 9 months, 10 months, 11 months, 12 months, or >12 months after the CAR-T cell therapy is administered.

If changes in the handwriting assessment indicate the development of agraphia, micrographia, and/or dysgraphia then the individual is subject to further neurologic assessment to evaluate neurotoxicity and identify/exclude non-CAR-T cell therapy causes of the observed neurotoxicity (e.g., infection). This neurologic assessment should include any one or more of the following: (i) assessment of a cerebral spinal fluid sample from the subject to analyze for the presence of infection, leptomeningeal disease, parananeoplastic syndrome, or a combination thereof; (ii) determination of the subject's serum levels of human herpes virus (HHV)-6, HHV-7, or both; (iii) measurement of the subject's serum thiamine levels; (iv) imaging the subject's brain via positron emission tomography or magnetic resonance imaging; and (v) performing an electroencephalogram (EEG).

Another aspect of the present application is directed to a method for treating multiple myeloma in a subject with chimeric antigen receptor (CAR) T cell therapy while minimizing neurotoxicity associated with said therapy. This method involves administering the CAR-T cell therapy to a subject having multiple myeloma, where the subject has a tumor burden characterized by bone marrow plasmacytosis of <80%, serum M Protein levels of <5 g/dL, and serum free light chain levels of <5000 mg/L.

In some embodiments, the subject has a tumor burden characterized by bone marrow plasmacytosis of <50%, serum M Protein levels of <3 g/dL, and serum free light chain levels of <3000 mg/L.

If the subject's tumor burden does not meet the requisite criteria for receiving the CAR-T cell therapy, e.g., because the subject's tumor burden is characterized by bone marrow plasmacytosis of >80%, serum M Protein levels of >5 g/dL, or serum free light chain levels of >5000 mg/L, then a bridging therapy can be administered to the subject in an amount effective to reduce the tumor burden in the subject to an acceptable level to receive the CAR-T cell therapy. For example, a bridging therapy is administered in an amount and duration effective to reduce the subject's tumor burden such that the subject's bone marrow plasmacytosis is <80%, serum M protein levels is <5 g/dL, and serum free light chain levels is <5000 mg/L. Suitable bridging therapeutics are described supra, and include, without limitation, chemotherapeutics (i.e., alkylating agents and topoisomerase inhibitors), immunomodulatory agents, protesome inhibitors, and any combination thereof.

Another aspect of the present disclosure is directed to a method for treating multiple myeloma in a subject with CAR-T cell therapy while reducing neurotoxicity associated with said therapy that involves administering the CAR-T cell therapy to a subject having multiple myeloma and an IL-6 serum level that is within the normal reference range of 0-2 pg/mL.

If the subject does not have an IL-6 serum level that falls within the normal reference range for IL-6 levels, then an IL-6 inhibitor therapy is administered prior to administering the CAR-T cell therapy in an amount and duration effective to reduce the IL-6 levels to normal reference levels. Suitable IL-6 inhibitors are described supra.

Another aspect of the present disclosure is directed to a method of reducing CAR-T cell therapy associated neurotoxicity in a subject receiving CAR-T cell therapy for the treatment of multiple myeloma. This method involves administering to the subject that has received CAR-T cell therapy and is having symptoms of CAR-T cell therapy associated cytokine release syndrome (CRS) or immune effector cell associated neurotoxicity syndrome (ICANS), an anti-inflammatory agent in an amount effective to reduce neurotoxicity in said subject. Suitable anti-inflammatory agents include, without limitation, IL-6 inhibitors (e.g., tocilizumab, sarliumab, siltuximab, or clazakizumab), IL-1 inhibitors (e.g., anakinra), Janus kinase 1/2 inhibitors (e.g., ruxolitinib) and corticosteroids, such as dexamethasone, methylprednisolone, hydrocortisone as described supra.

Another aspect of the present disclosure is directed to a method of reducing CAR-T cell therapy associated neurotoxicity in a subject receiving CAR-T cell therapy for the treatment of multiple myeloma. This method involves administering, to a subject that has received CAR-T cell therapy and has a high level of CAR-T cell expansion a chemotherapeutic to reduce the level of CAR-T cells in the subject and the associated neurotoxicity. As described supra, a high level of CAR-T cell expansion is defined as a maximum plasma concentration (Cmax) of >1,000 cells/μL. Similarly, if the subject has received CAR-T cell therapy and has a high level of CAR-T cell persistence, a chemotherapeutic agent is administered to the subject to reduce the persisting CAR-T cell levels and associated neurotoxicity. As described supra, a high level of CAR-T cell persistence is defined as a peripheral blood concentration of >300 cells/μL at about 45-65 days after receiving the CAR-T cell therapy.

Another aspect of the present disclosure is directed to a method of reducing CAR-T cell therapy associated neurotoxicity in a subject receiving CAR-T cell therapy for the treatment of multiple myeloma. This method involves administering, to a subject that has received CAR-T cell therapy and has developed an increase in peripheral blood IL-6 level that is above the upper limit of normal IL-6 levels, an IL-6 inhibitor to reduce CAR-T cell therapy associated neurotoxicity. Suitable IL-6 inhibitors are described supra.

Another aspect of the present disclosure is directed to a method of reducing CAR-T cell therapy associated neurotoxicity in a subject receiving CAR-T cell therapy for the treatment of multiple myeloma. This method involves administering, to a subject that has received CAR-T cell therapy and has developed an increase in peripheral blood INF-γ level that is above the upper limit of normal INF-γ levels, an INF-γ inhibitor to reduce CAR-T cell therapy associated neurotoxicity. Suitable INF-γ inhibitors are described supra.

### EXAMPLES

### Example 1—Clinical Study 68284528MMY2001

### Study Design

Study 68284528MMY2001 was a Phase 1b-2, open-label, multicenter study designed to evaluate the safety and efficacy of cilta-cel in adult subjects with relapsed or refractory multiple myeloma (RRMM). The study was comprised of 2 parts: Phase 1b and Phase 2. In the Phase 1b portion, a staggered enrollment strategy was used to confirm the recommended dose level for investigation in Phase 2 (RP2D). In the Phase 2 portion, additional subjects were treated with cilta-cel using the RP2D to further characterize safety and efficacy.

Enrolled subjects underwent apheresis to acquire peripheral blood mononuclear cells (PBMC), and cilta-cel was prepared using the subject's T-cells selected from the apheresis product. After cilta-cel production and product release, subjects received a 3-day lymphodepletion conditioning regimen of cyclophosphamide and fludarabine, followed by cilta-cel infusion 5 days to 7 days after the start of conditioning. Some study subjects received bridging therapy between apheresis and the start of lymphodepletion chemotherapy to maintain disease stability. Subjects were monitored closely for safety and disease assessments during the period following cilta-cel infusion (Day 1 to Day 100). Assessments during the post-treatment period (Day 101 to study completion) were less frequent, with safety and disease assessments performed every 28 days. Survival status and subsequent anti-cancer therapy information was collected every 16 weeks following disease progression. The study will be completed 2 years after the last subject has received his or her initial dose of cilta-cel. Thereafter, subjects treated with cilta-cel are to be enrolled in a long-term follow-up study (Study 68284528MMY4002) for continued monitoring for up to 15 years.

### Safety Analysis Set

The primary analysis population for all safety analyses was the all-treated population which included all 97 subjects who received a cilta-cel infusion as of the clinical cutoff date.

### Chimeric Antigen Receptor T-Cell (CAR-T) Neurotoxicity

CAR-T neurotoxicity is categorized as immune effector cell-associated neurotoxicity syndrome (ICANS) and/or Other Neurotoxicity related to CAR-T therapy and occurring after recovery from cytokine release syndrome (CRS) and/or ICANS. Other Neurotoxicities were categorized as movement and neurocognitive adverse events.

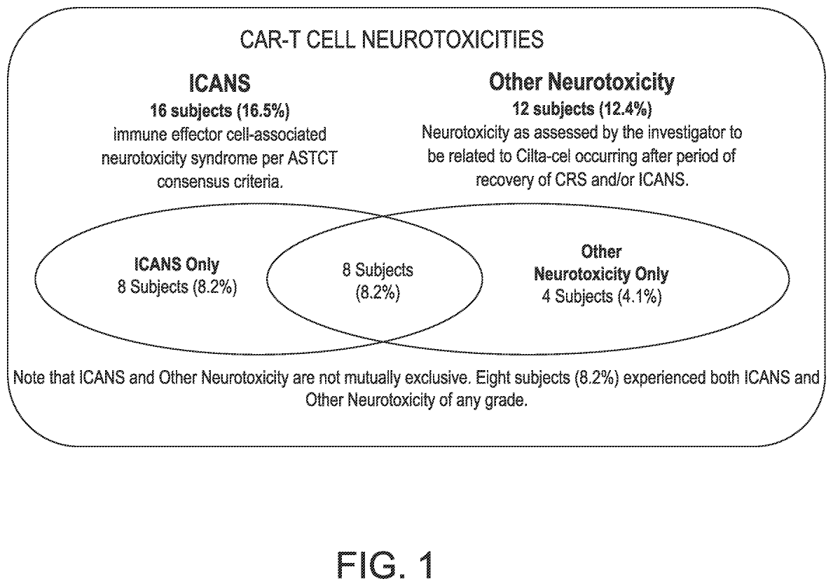

In Study 68284528MMY2001, 20 subjects (20.6%) experienced a treatment-emergent CAR-T neurotoxicity event. Nine subjects (9.3%) experienced a Grade 3 or 4 event and 1 subject (1.0%) experienced a Grade 5 event. Note that ICANS and Other Neurotoxicities are not mutually exclusive as 8 subjects (8.2%) experienced both ICANS and Other Neurotoxicity of any grade as are depicted in FIG. 1.

### Immune Effector Cell-Associated Neurotoxicity

Sixteen subjects (16.5%) experienced ICANS (Table 1). Ten subjects (10.30%) had a maximum Grade 1 event; 4 subjects (4.10%) had a maximum Grade 2 event; and 1 subject (1.00%) each a maximum Grade 3 or Grade 4 event. No subjects experienced a Grade 5 ICANS event.

TABLE 1
Summary of Immune Effector Cell-Associated Neurotoxicity
With Onset After Cilta-cel Infusion; All Treated Analysis Set
(Study 68284528MMY2001)

| | | | | | Phase 1b + | |
|---|---|---|---|---|---|---|
| | Phase 1b | | Phase 2 | | Phase 2 | |
| Analysis set: all treated | 29 | | 68 | | 97 | |
| Number of subjects | 3 | (10.3%)^{a} | 13 | (19.1%) | 16 | (16.5%) |
| with ICANS | | | | | | |
| Maximum | | | | | | |
| toxicity grade | | | | | | |
| Grade 1 | 2 | (6.9%) | 8 | (11.8%) | 10 | (10.3%) |
| Grade 2 | 0 | | 4 | (5.9%) | 4 | (4.1%) |
| Grade 3 | 1 | (3.4%) | 0 | | 1 | (1.0%) |
| Grade 4 | 0 | | 1 | (1.5%) | 1 | (1.0%) |
| Grade 5 | 0 | | 0 | | 0 | |
| Time from initial | | | | | | |
| infusion of Cilta-cel to | | | | | | |
| first onset of ICANS | | | | | | |
| N | 3 | | 13 | | 16 | |
| Mean (SD) | 6.3 | (2.89) | 7.5 | (2.22) | 7.3 | (2.29) |
| Median | 8.0 | | 8.0 | | 8.0 | |
| Range | (3; 8) | | (4; 12) | | (3; 12) | |
| Duration | | | | | | |
| of ICANS (days) | | | | | | |
| N | 3 | | 13 | | 16 | |
| Mean (SD) | 3.7 | (2.08) | 5.2 | (3.09) | 4.9 | (2.93) |
| Median | 3.0 | | 4.0 | | 4.0 | |
| Range | (2; 6) | | (1; 12) | | (1; 12) | |
| Number of subjects | 3 | (10.3%) | 13 | (19.1%) | 16 | (16.5%) |
| with treatment of | | | | | | |
| ICANS | | | | | | |
| IL-1 receptor | 0 | | 3 | (4.4%) | 3 | (3.1%) |
| antagonist anakinra | | | | | | |
| Anti-IL6 receptor | 1 | (3.4%) | 3 | (4.4%) | 4 | (4.1%) |
| tocilizumab | | | | | | |
| Corticosteroid | 1 | (3.4%) | 8 | (11.8%) | 9 | (9.3%) |
| Levetiracetam | 0 | | 2 | (2.9%) | 2 | (2.1%) |
| Dexamethasone | 1 | (3.4%) | 8 | (11.8%) | 9 | (9.3%) |
| Methylprednisolone | 0 | | 1 | (1.5%) | 1 | (1.0%) |
| sodium succinate | | | | | | |
| Pethidine | 0 | | 1 | (1.5%) | 1 | (1.0%) |
| Outcome of ICANS | | | | | | |
| N | 3 | | 13 | | 16 | |
| Recovered or resolved | 3 | (100.0%) | 13 | (100.0%) | 16 | (100.0%) |
| Concurrent CRS | | | | | | |
| Yes | 3 | (100.0%) | 12 | (92.3%) | 15 | (93.8%) |

| No | 0 | 1 | (7.7%) | 1 | (6.3%) |
|---|---|---|---|---|---|
| ICANS prior to CRS | 0 | 0 | | 0 | |
| ICANS following CRS | 0 | 1 | (7.7%) | 1 | (6.3%) |

CRS = Cytokine Release Syndrome,
ICANS = Immune Effector Cell-Associated Neurotoxicity,
TE = treatment-emergent.
^{a}For 2 subjects in Phase 1b, the reported term is CAR-T cell Related Encephalopathy Syndrome (CRES). These events were reported prior to publication of the ASTCT consensus grading system and graded according to NCI-CTCAE version 5.0. For these 2 subjects, the maximum toxicity grade was Grade 1 and Grade 3, respectively according to NCI-CTCAE version 5.0
Note:
ICANS evaluated according to the ASTCT consensus grading system (Lee et al 2019) or NCI-CTCAE version 5.0.
Note:
Percentages are calculated with the number of subjects in the all treated analysis set as denominator, except for the outcome of ICANS and concurrent CRS for which percentages are calculated with the number of subjects with ICANS in the all treated analysis set as denominator.
Note:
Treatments for ICANS include treatments administered for ICANS and symptoms of ICANS.
Note:
ICANS and CRS are considered to be concurrent if there is an overlap in the duration of these respective events.

For 2 subjects in Phase 1b, the reported term was CAR-T related encephalopathy syndrome (CRES), coded to ICANS according to the Medical Dictionary for Regulatory Activities (MedDRA v 23), in Table 2. These events were reported prior to publication of the American Society for Transplantation and Cellular Therapy (ASTCT) consensus criteria and were, therefore, graded according to the National Cancer Institute-Common Terminology Criteria for Adverse Events (NCI-CTCAE v 5.0) and are included as ICANS in Table 1. For those 2 subjects, the maximum grade was Grade 1 (1 subject) and Grade 3 (1 subject) according to NCI-CTCAE v 5.0.

TABLE 2
Summary of Other Neurotoxicities; All Treated Analysis Set (Study
68284528MMY2001)

| | Phase 1b + Phase 2 | |
|---|---|---|
| Analysis set: all treated | 97 | |
| Number of subjects with other neurotoxicities | 12 | (12.4%) |
| Maximum toxicity grade | | |
| Grade 1 | 0 | |
| Grade 2 | 3 | (3.1%) |
| Grade 3 | 7 | (7.2%) |
| Grade 4 | 1 | (1.0%) |
| Grade 5 | 1 | (1.0%) |
| Time from initial infusion of Cilta-cel to first | | |
| onset of other neurotoxicities | | |
| N | 12 | |
| Mean (SD) | 42.8 | (36.64) |
| Median | 26.5 | |
| Range | (11; 108) | |
| Time to recovery of other neurotoxicities^{a} (days) | | |
| N | 5 | |
| Mean (SD) | 67.6 | (59.92) |
| Median | 70.0 | |
| Range | (2; 159) | |
| Outcome of other neurotoxicities | | |
| N | 12 | |
| Recovered or resolved | 5 | (41.7%) |
| Not recovered or not resolved | 5 | (41.7%) |
| Recovering or resolving | 1 | (8.3%) |
| Fatal | 1 | (8.3%) |

^{a}Calculated for other neurotoxicities with outcome resolved/recovered.
Note:
Percentages are calculated with the number of subjects in the all-treated analysis set as denominator, except for the outcome of other neurotoxicities for which percentages are calculated with the number of subjects with other neurotoxicities in the all-treated analysis set as denominator.

The median time from cilta-cel infusion to first onset of ICANS was 8.0 days (range: 3 to 12 days) and the median duration was 4 days (range: 1 to 12 days). Treatment-emergent symptoms of clinical note for ICANS included aphasia, slow speech, dysgraphia, encephalopathy, depressed level of consciousness, and confusional state.

At the time of clinical cutoff, all 16 subjects who had experienced ICANS had recovered. All subjects who experienced ICANS also experienced CRS. Fifteen subjects experienced ICANS concurrent with CRS and 1 subject experienced ICANS 4 days after the recovery of CRS.

### Other Neurotoxicities

Twelve subjects (12.4%) experienced other CAR-T neurotoxicities not defined as ICANS as assessed by the Investigator either due to the symptoms or time of onset (i.e., occurring after a period of recovery from CRS and/or ICANS). These events included a variety of symptoms with varying severity including disturbances in consciousness, coordination and balance disturbances, movement and motor dysfunction, mental impairment TEAEs, cranial nerve disorders, and peripheral neuropathies.

Three subjects (3.1%) experienced a maximum toxicity of Grade 2. Eight subjects (8.2%) experienced Grade 3 or 4 toxicities, and 1 subject (1.0%) experienced a Grade 5 toxicity. These events had a median onset of 26.5 days from cilta-cel infusion (range 11 to 108 days) with a median time to recovery of 70.0 days (range 2 to 159 days). At the time of clinical cutoff, 5 of these 12 cases (41.7%) resolved, 5 cases (41.7%) did not resolve, 1 case (8.3%) was recovering/resolving, and 1 case (8.3%) was fatal due to Grade 5 neurotoxicity. These events are summarized in Table 2.

### Other Neurotoxicities Characterized by Movement and Neurocognitive Treatment-Emergent Adverse Events

The symptoms associated with other CAR-T neurotoxicity events varied widely in 12 subjects. However, 5 of the 12 subjects (Subjects L28US10002023, L28US10003011, L28US10017023, L28US10021005, and L28US10025003) experienced a similar presentation of movement and neurocognitive treatment-emergent adverse events (TEAEs). These included a cluster of movement (e.g., micrographia, tremors, etc.), cognitive (e.g., memory loss, disturbance in attention, etc.) and changed personality (e.g., reduced facial expression, flat affect, etc.) TEAEs; in some instances, these TEAEs progressed to an inability to work or care for oneself.

One subject (1.0%) experienced a maximum toxicity of Grade 2. Three subjects (3.1%) experienced Grade 3 toxicities, and 1 subject (1.0%) experienced a Grade 5 toxicity. These events had a median onset of 27 days from cilta-cel infusion (range 14 to 108 days) (Table 3). Subjects were treated with steroids, systemic chemotherapy (cyclophosphamide), intrathecal chemotherapy (methotrexate, cytarabine), IL-1 receptor antagonist (anakinra), tyrosine kinase inhibitor (dasatinib), anti-IL-6 antibody (siltuximab), and other agents (e.g., carbidopa/levodopa, levetiracetam, etc.) with limited or no observed improvement in symptomatology.

At the time of clinical cutoff, 1 (8.3) case was recovering/resolving, 1 case (8.3) was fatal due to Grade 5 neurotoxicity, and 3 (25.0%) cases had not recovered/not resolved (of which 2 cases were fatal due to other causes [lung abscess and septic shock, both confirmed by autopsy]) (Table 3).

TABLE 3
Summary of Movement and Neurocognitive Toxicities; All Treated
Analysis Set (Study 68284528MMY2001)

| | Movement and | |
|---|---|---|
| | Neurocognitive | |
| | Treatment-Emergent | |
| | Adverse Events | |
| | Phase 1b + Phase 2 | |
| Analysis set: all treated | | |
| Number of subjects with other neurotoxicities | 5 | (5.2%) |
| Maximum toxicity grade | | |
| Grade 1 | 0 | |
| Grade 2 | 1 | (1.0%) |
| Grade 3 | 3 | (3.1%) |
| Grade 4 | 0 | |
| Grade 5 | 1 | (1.0%) |
| Time from initial infusion of Cilta-cel to | | |
| first onset of other neurotoxicities | | |
| N | 5 | |
| Mean (SD) | 53.0 | (47.36) |
| Median | 27.0 | |
| Range | (14; 108) | |
| Time to recovery of other neurotoxicities^{a} (days) | | |
| N | 0 | |
| Mean (SD) | — | |
| Median | — | |
| Range | — | |
| Outcome of other neurotoxicities | | |
| N | 5 | |
| Recovered or resolved | 0 | |
| Not recovered or not resolved | 3 | (25.0%) |
| Recovering or resolving | 1 | (8.3%) |
| Fatal | 1 | (8.3%) |

^{a}Calculated for other neurotoxicities with outcome resolved/recovered.
Note:
Percentages are calculated with the number of subjects in the all-treated analysis set as denominator, except for the outcome of other neurotoxicities for which percentages are calculated with the number of subjects with other neurotoxicities in the all-treated analysis set as denominator.

The presentation of movement, neurocognitive, and personality change TEAEs in these 5 subjects appears to be potentially associated with a combination of 2 or more factors such as high tumor burden, prior Grade 2 or higher CRS, prior ICANS, and high CAR-T cell expansion and persistence. To minimize the risk for neurotoxicity for subjects in the ongoing cilta-cel clinical development program, monitoring and mitigation strategies were implemented including: 1) enhanced bridging therapy to reduce baseline tumor burden; 2) early aggressive treatment of CRS and ICANS; 3) handwriting assessments for the early detection of neurotoxicity symptoms; and 4) an extended monitoring and reporting time for neurotoxicity up to one-year post-cilta-cel infusion. Thereafter, subjects treated with cilta-cel are to be enrolled in a long-term follow-up study (Study 68284528MMY4002) for continued monitoring for up to 15 years.

### Example 2—Clinical Study 68284528MMY2003

Study 68284528MMY2003 is a Phase 2, multicohort, open-label, multicenter study to determine whether treatment with cilta-cel results in minimal residual disease (MRD) negativity in adult subjects with multiple myeloma (MM). Cohorts of approximately 20 subjects each, representing unique patient populations with MM and with an unmet medical need, are planned.
Cohort A=Progressive disease after 1 to 3 prior lines of therapy (including a proteasome inhibitor (PI) and an immunomodulatory agent (IMiD)) and refractory to lenalidomide.
Cohort B=1 prior line of therapy containing a PI and IMiD and early relapse defined as disease progression <12 months after an autologous stem cell transplant (ASCT) or <12 months after the start of front-line therapy for subjects who have not had an ASCT.
Cohort C=Relapsed or refractory disease in subjects previously treated with a PI, IMiD, anti-CD38 monoclonal antibody (mAb), and BCMA-directed therapy (excluding immunotherapy).
Cohort D=Cilta-cel plus lenalidomide. Subjects with MM without a complete response (CR) after 4 to 8 total cycles of initial therapy, including induction, high-dose chemotherapy, and ASCT with or without consolidation.
Cohort E=Daratumumab, bortezomib, lenalidomide, and dexamethasone (D-VRd) induction, Cilta-cel, then daratumumab and lenalidomide (D-R). Subjects with high risk newly diagnosed and untreated MM (hr-NDMM) for whom an ASCT is not planned as initial therapy.

Eligible subjects are to undergo apheresis, PBMC collection, and CAR-T generation as described for Study 8284528MMY2001 (Example 1). Strategies to mitigate movement and neurocognitive TEAEs based on the findings from Study 68284528MMY2001 were implemented in Study 68284528MMY2003.

Other Neurotoxicity events not defined as ICANS (as assessed due to symptoms or time of onset), were reported for 2 subjects (11.1%). These Other Neurotoxicity events included slow speech, facial paralysis, gait disturbance, and pain, each reported at an incidence of 5.6%. These events were Grade 1 or 2 severity, and none were considered serious. The median time to onset was 20.0 days (range: 11 to 29 days). For 1 subject, the events resolved within 4 days and were ongoing for the other subject.

After the clinical cutoff for Study 68284528MMY2003, a case of Other Neurotoxicity was reported for one subject in Cohort B, and the adverse events included movement and neurocognitive TEAEs. The subject is a male aged 44 years. He was dosed with cilta-cel infusion on Study Day 1. The subject experienced CRS between Study Days 6 through 10, with a maximum Grade 3 severity. The subject was treated with tocilizumab, dexamethasone, dopamine, norepinephrine, and antibiotics. The CRS resolved, and the subject was discharged on Study Day 16. The subject did not experience ICANS.

On Study Day 55, the subject experienced encephalopathy with frontal lobe symptoms (Grade 3) and bradykinesia (Grade 3). Flat affect, akinetic mutism, apathy, release signs (mild to moderate), symmetric rigidity, and Parkinson's type gait and stance (without tremor) were observed. The subject was hospitalized. Evaluations included an MRI of the brain with subtle abnormalities of the bilateral caudate noted (i.e., hyperintensity on fluid attenuated inversion recovery (FLAIR) sequences), an EEG with bilateral temporal lobe slowing, PET/CT brain scans with results pending, and CSF assessment for infectious diseases and paraneoplastic antibody panel tests that were negative. The subject was treated with high-dose methylprednisolone, plasmapheresis, and IV immunoglobulin. The events are ongoing and considered related to cilta-cel.

### Example 3—Clinical Study 68284528MMY3002

Study 68284528MMY3002 is a Phase 3, randomized study comparing cilta-cel versus pomalidomide, bortezomib, and dexamethasone or daratumumab, pomalidomide, and dexamethasone in subjects with relapsed and lenalidomide-refractory MM. Approximately 400 subjects are planned (200 subjects per treatment arm). Strategies to mitigate movement and neurocognitive TEAEs based on the findings from Study 68284528MMY2001 (Example 1) were implemented in Study 68284528MMY3002.

As of the clinical cutoff, 1 of 16 subjects treated with cilta-cel experienced Other Neurotoxicities. The subject experienced left facial paralysis (Study Day 18) and subsequent left eye diplopia (Study Day 36). Both events were Grade 2 in severity, and both resolved (Study Day 56 and Study Day 53, respectively). No subject in this study has reported movement or neurocognitive TEAEs.

After the clinical cutoff for Study 68284528MMY3002, two cases of Other Neurotoxicity were reported. One subject experienced left Bell's palsy (Post-infusion Day 24). The event was Grade 2 in severity and is ongoing. Patient received treatment with Prednisone 60 mg daily for 3 days with taper over an additional 8 days. This patient also experienced problems naming words and long-term memory problems starting on Post-infusion Day 34 and 42 respectively. Both of these Grade 1 events are ongoing. One subject experienced bilateral facial palsy (Post-infusion Day 25). The event was Grade 2 in severity and is ongoing. MRI brain showed no acute intracranial abnormality. It did demonstrate CSF cytology showed mature lymphocytes and monocytes. CSF flow cytometry showed >95% CD5 positive presumptive T cells and no evidence of malignancy. CSF meningitis/encephalitis panel was negative. CSF cultures, cryptococcal antigen and AFB smear were negative. Patient received treatment with Prednisone 60 mg daily for 2 days with taper over an additional 8 days. No movement or neurocognitive TEAEs have been reported in this study to date.

### Example 4—Evaluation of Factors Contributing to CAR-T Cell Neurotoxicity

Neurotoxicity after resolution of CRS has been reported in the literature for CD19 targeted chimeric antigen receptor T-cell (CAR T) therapies. In addition, a case of Grade 3 parkinsonism has been referenced in the USPI for ide-cel (a BCMA targeted CAR-T therapy). It was noted that in the 68284528MMY2001 study, there are cases of Other Neurotoxicities characterized by movement and neurocognitive TEAEs that occurred after a period of recovery from cytokine release syndrome (CRS) or immune effector cell-associated neurotoxicity syndrome (ICANS) which appeared to share common features in terms of clinical presentation and CAR-T correlative data. Similar to those observed in the approved CD19 and B-cell maturation antigen (BCMA) directed CAR-T therapies, the exact mechanism of this neurotoxicity is not clear at this time. Serial brain magnetic resonance imaging (MRIs) and virology workups so far have been essentially negative in these cases. In addition, comprehensive paraneoplastic and autoimmune antibody panel evaluations have been negative, where performed.

Objective. The primary objective of this analysis was to explore the clinical data in relationship to the onset of movement and neurocognitive TEAEs in patients with multiple myeloma who received a cilta-cel infusion.

Method. An analysis was conducted to explore the clinical data including: demographics, baseline disease characteristics, baseline clinical laboratory values, exposure to cilta-cel, adverse events of CRS or ICANS (including grades), lymphocyte counts, neutrophil counts, and platelet counts in relationship to the onset of movement and neurocognitive TEAEs.

Subjects and Database. Ninety-seven subjects who received a cilta-cel infusion from the main cohort in Study 68284528MMY2001 were included in the analysis. Five subjects were considered as having movement and neurocognitive TEAEs.

Study Day and Baseline. Study Day 1 refers to the start of the initial administration of cilta-cel. The baseline value is defined as the closest non-missing value before the initial dose of cilta-cel (including time if time is available), with the exception of parameters associated with disease-related efficacy assessments for which the baseline value is defined as the non-missing value closest to the start of the conditioning regimen and before cilta-cel infusion. Depending on the protocol time and event schedule, baseline for non-efficacy variables can be at screening, prior to conditioning, or prior to cilta-cel infusion.

Movement and Neurocognitive TEAEs. Subjects with Other Neurotoxicities characterized by movement and motor dysfunction TEAE and cognitive impairment TEAEs were identified by the time of onset of the CAR-T related neurotoxicities (i.e., after recovery of CRS and/or ICANS) and the reporting of MedDRA preferred terms (Table 4) from among at least two of the following categories:

TABLE 4
Neurotoxicity Categories

| Category | Preferred Term |
|---|---|
| Movement | Ataxia, Balance disorder, Bradykinesia, Cogwheel rigidity, |
| and Motor | Dysgraphia, Dyskinesia, Dysmetria, Essential tremor, Gait |
| Dysfunction | disturbance, Hand-eye coordination impaired, Micrographia, |
| TEAE | Motor dysfunction, Myoclonus, Parkinsonism, Posture |
| | abnormal, Resting tremor, Stereotypy, Tremor |
| Cognitive | Amnesia, Apraxia, Bradyphrenia, Cognitive disorder, |
| Impairment | Confusional state, Depressed level of consciousness, |
| TEAE | Disturbance in attention, Encephalopathy, Incoherent, |
| | Leukoencephalopathy, Loss of consciousness, Memory |
| | impairment, Mental impairment, Mental status changes, |
| | Noninfective encephalitis, Psychomotor retardation |
| Personality | Flat affect, Personality change, Reduced facial expression |
| changes | |

Statistical Methods. Descriptive statistics and frequency distribution with the number and percentage of subjects in each category were included as appropriate. Differences between subjects with and without movement and neurocognitive TEAEs in the clinical variables were assessed using Wilcoxon rank sum test for continuous variables and Fisher's Exact test for categorical variables. Logistic regression models with movement and neurocognitive TEAEs as dependent variable were performed to evaluate each of the clinical variables as an independent variable.

Given the exploratory nature of the analysis with multiplicity and the small number of events (i.e., movement and neurocognitive TEAEs), results were intended for hypothesis generating and should be interpreted with caution.

Data Handling. Cytokine: Central lab data from FRONTAGE were used. For results that were “<lower limit of quantification (LLOQ)”, LLOQ was used in this analysis.

### Result

The variables explored in the search of common features in subjects who presented with movement or neurocognitive TEAEs included the following.

### Baseline Variables:

Demographics: age, sex, race, ethnicity;
Baseline tumor burden: bone marrow plasmacytosis, LDH, soft tissue plasmacytoma (extramedullary (Y/N, count, SPD)), serum M-spike/serum free light chain;
Baseline disease characteristics: disease tempo (percent change of tumor burden from screening to baseline); type of multiple myeloma, measurable disease type, lines of prior therapy, time since MM diagnosis, ECOG PS, tumor BCMA expression, time from last prior to apheresis;
Bridging therapy use, bridging daratumumab/lenalidomide use;
Viral infection prior to apheresis, prior radiotherapy of the brain;
Baseline laboratory values (fibrinogen, C-reactive protein, ferritin, platelet counts, β-2 microglobulin, IL-6, IL-10, INF-γ, IL-2 receptor a, and estimated glomerular filtration rate); and
Exposure data: dose administered, infusion duration.

### Post-Baseline Variables:

CRS, CRS max grade (<2 vs ≥2), ICANS, ICANS max grade (<2 vs ≥2), concomitant therapies for CRS and ICANS (i.e., steroid, tocilizumab, anakinra);
Absolute lymphocyte counts (ALC), absolute neutrophil counts (ANC), platelet counts in first 30 days from local complete blood count (CBC);
Maximum ferritin levels in first 2/3 weeks, ferritin at day 28; and
CAR-T cell expansion, CAR-T cell persistence.
   Other variables explored included patterns of BCMA expression in normal (non-diseased) human brain sections as well as the cilta-cel dose and manufacturing controls.

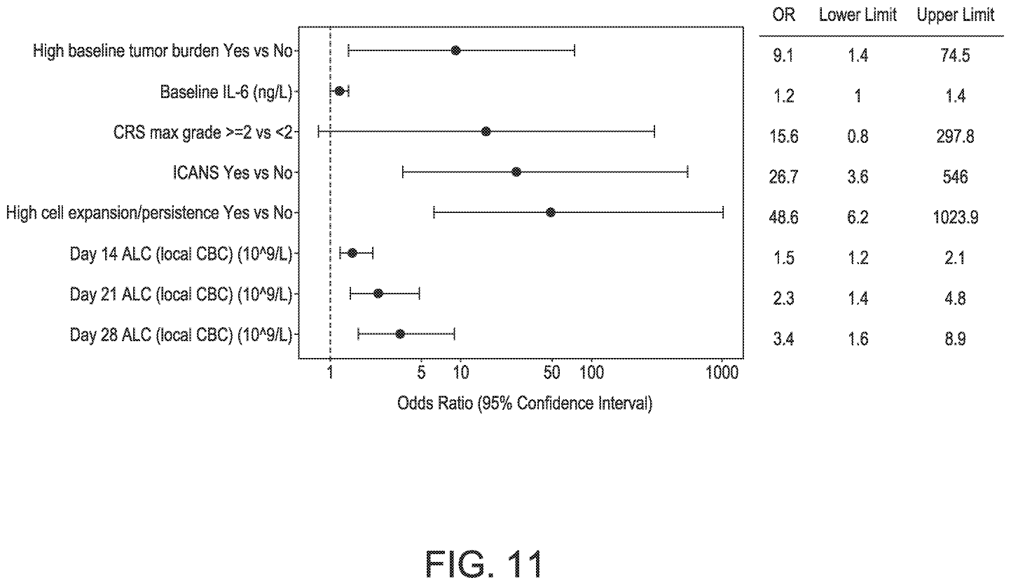

Analysis of these variables is discussed in detail below. Contingency tables and boxplots for evaluation of the correlation between the clinical variables and onset of movement and neurocognitive TEAEs are presented. In summary, there appeared to be an association between the following variables and the onset of movement and neurocognitive TEAEs: high tumor burden at baseline, baseline IL-6, CRS maximum grade, ICANS, lymphocytes on Days 14, 21, 28, and high cell expansion/persistence. A forest plot of estimated odds ratios (ORs) and confidence intervals (CIs) for these variables in relationship to onset of movement and neurocognitive TEAEs is provided (FIG. 11).

### Demographics

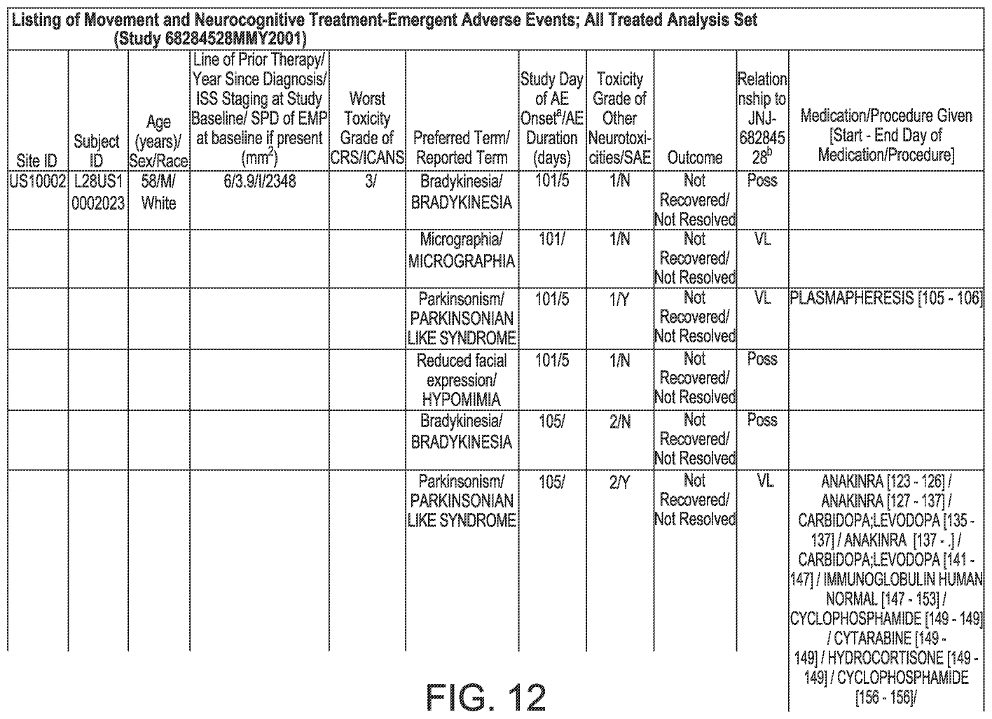

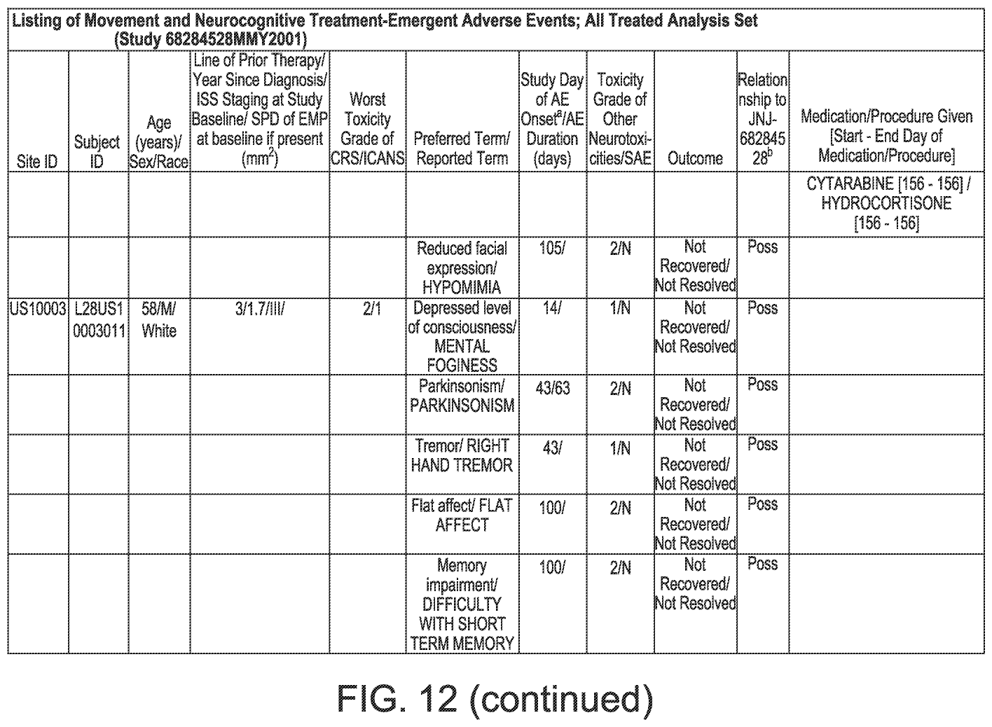

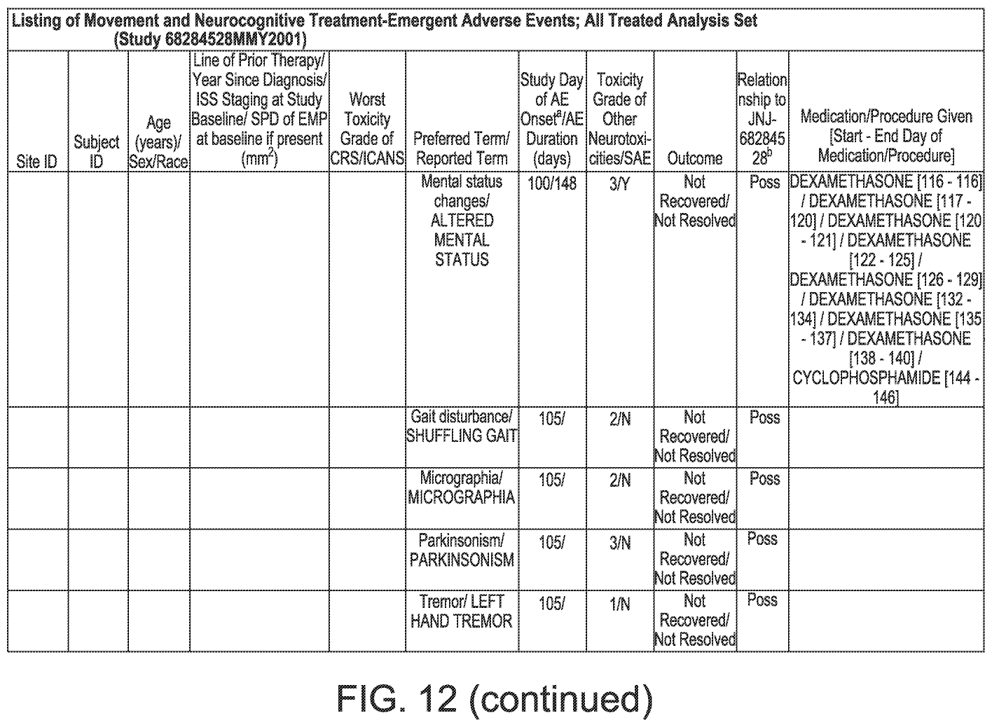

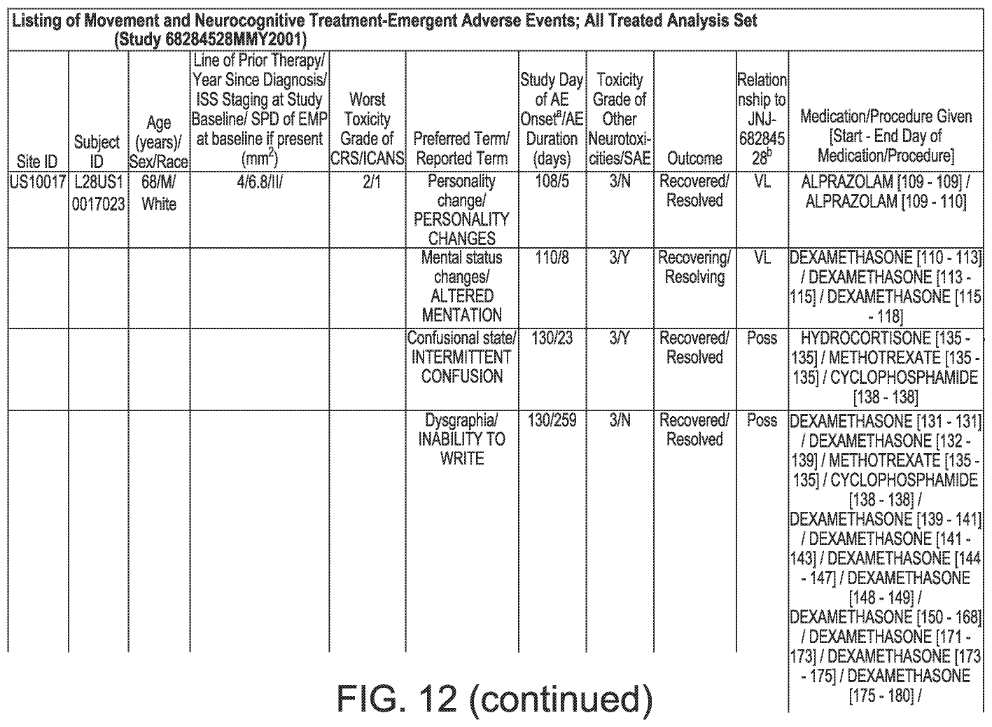

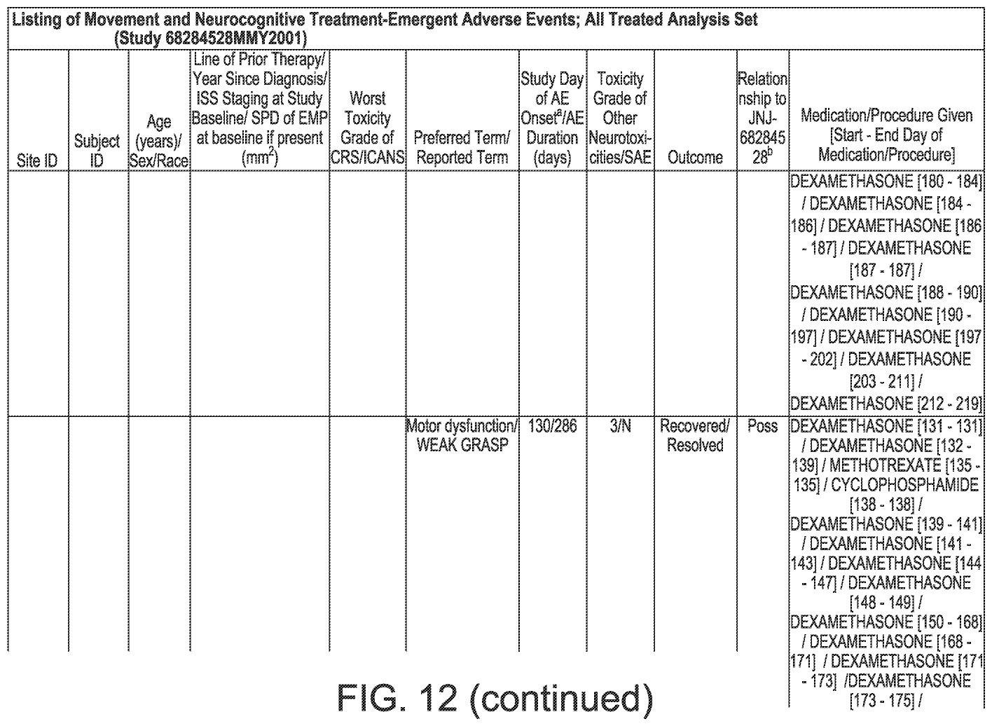

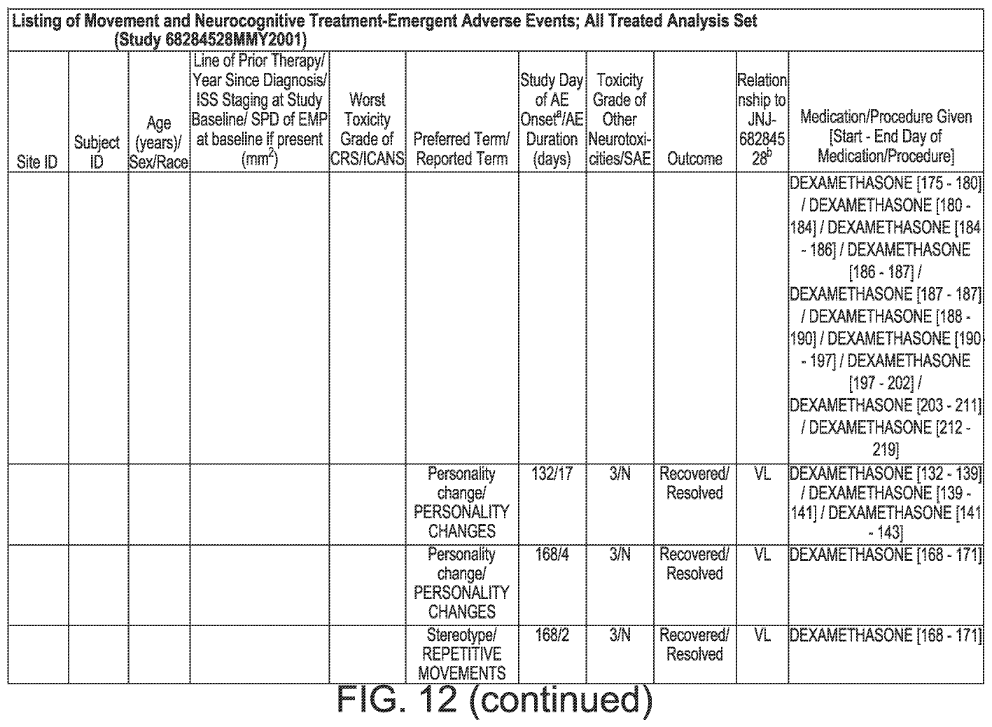

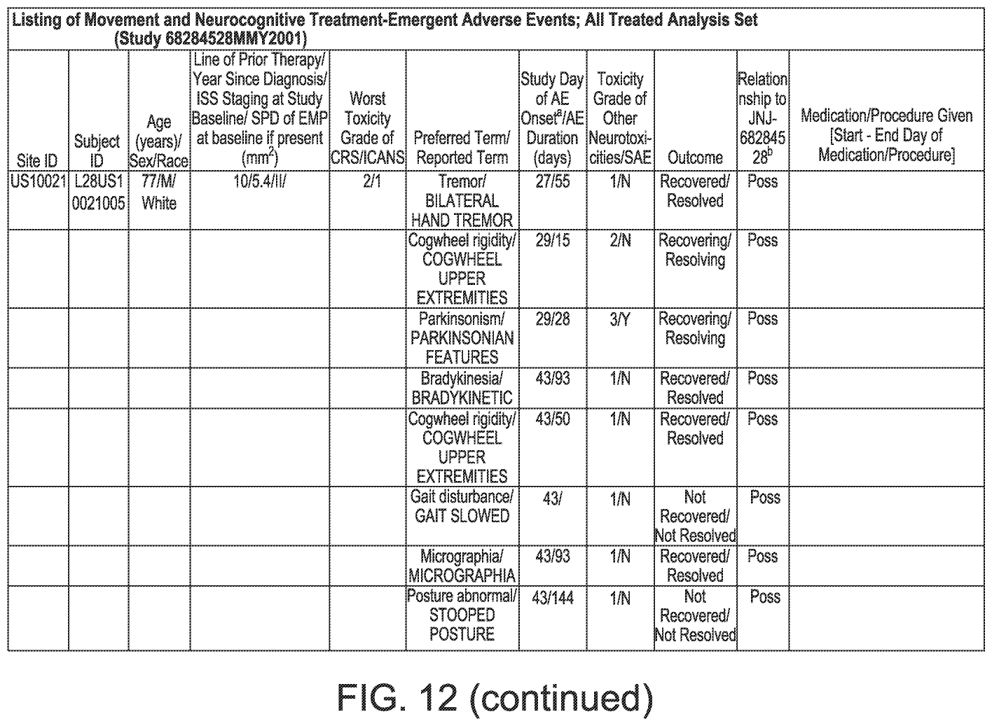

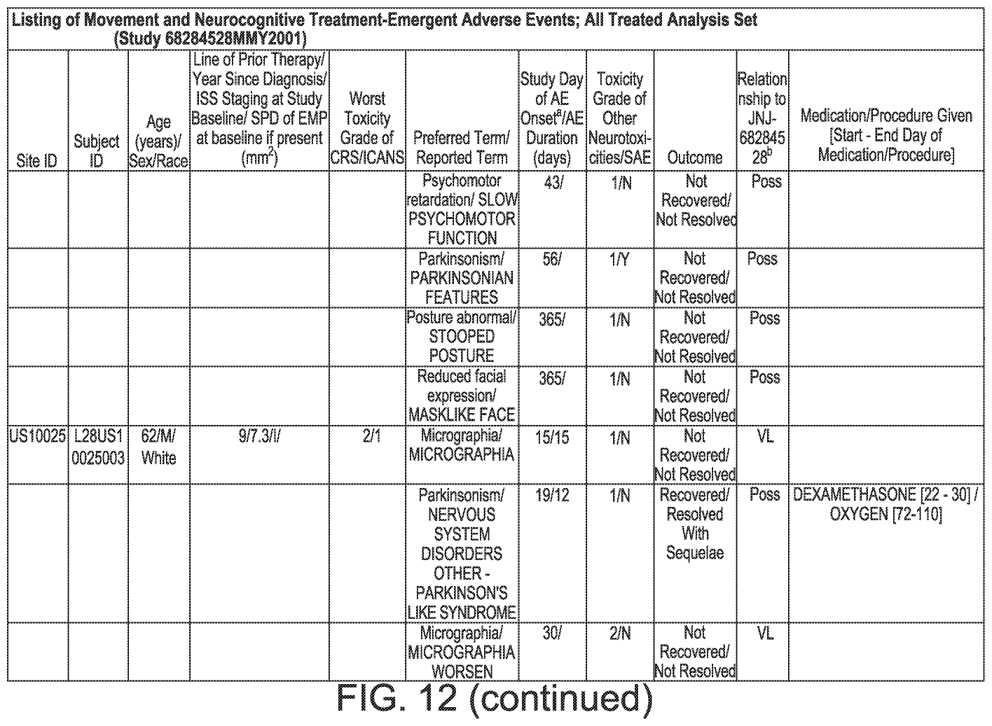

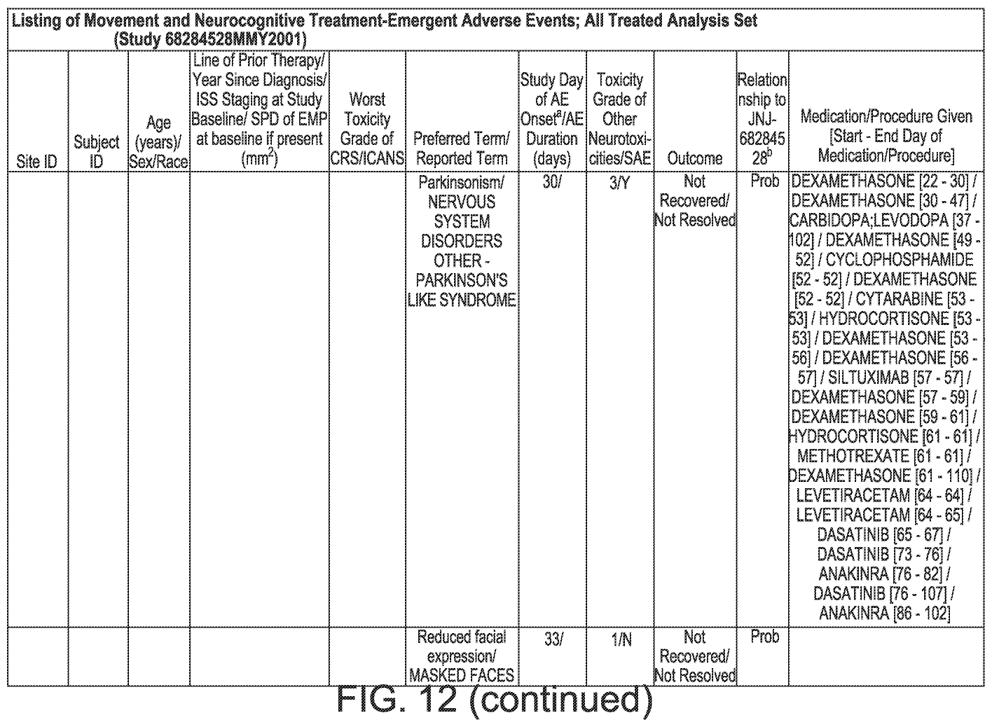

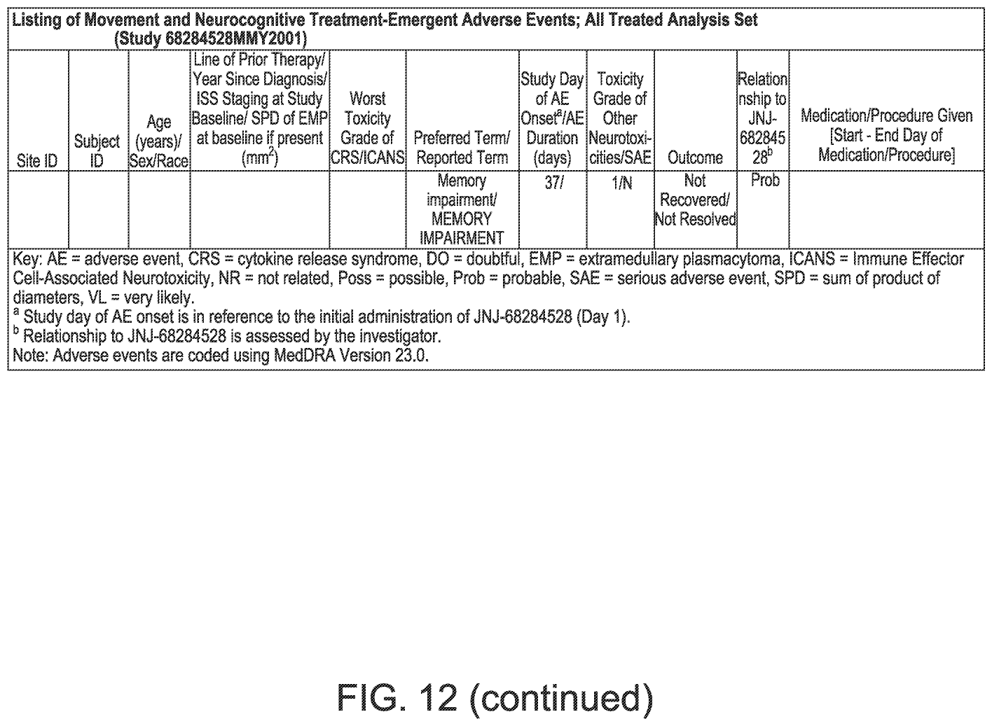

A possible relationship between the subject demographics and the occurrence of the observed movement and neurocognitive TEAEs was investigated. Of the 5 subjects in Study 68284528MMY2001 who met the criteria defining movement and neurocognitive TEAEs including changes in personality, all 5 were white males (Table 5 and FIG. 12).

TABLE 5
Subject Demographics and Baseline Characteristics;
All Treated Analysis Set (Study 68284528MMY2001)

| | Movement and Neurocognitive | | | | |
|---|---|---|---|---|---|
| | Treatment-Emergent Adverse Events | | | | |
| | No | | Yes | | Total |
| | N = 92 | | N = 5 | | N = 97 |
| Age Group (years) | n (%) | | n (%) | | |
| <65 | 59 | (64.1) | 3 | (60.0) | 62 |
| 65-75 | 26 | (28.3) | 1 | (20.0) | 27 |
| >75 | 7 | (7.6) | 1 | (20.0) | 8 |
| Sex | | | | | |
| Female | 40 | (43.5) | 0 | (0.0) | 40 |
| Male | 52 | (56.5) | 5 | (100.0) | 57 |
| Race Group | | | | | |
| White | 64 | (69.6) | 5 | (100.0) | 69 |
| African American | 17 | (18.5) | 0 | (0.0) | 17 |
| Other | 11 | (12.0) | 0 | (0.0) | 11 |
| Ethnicity | | | | | |
| Hispanic or Latino | 6 | (6.5) | 0 | (0.0) | 6 |
| Non-Hispanic or Latino | 80 | (87.0) | 5 | (100.0) | 85 |
| Not Reported | 6 | (6.5) | 0 | (0.0) | 6 |
| ECOG PS prior to Cilta-cel | | | | | |
| infusion^{a} | | | | | |
| 0 | 36 | (39.1) | 3 | (60.0) | 39 |
| 1 | 52 | (56.5) | 2 | (40.0) | 54 |
| 2 | 4 | (4.3) | 0 | (0.0) | 4 |

cilta-cel = ciltacabtagene autoleuce;
ECOG PS = Eastern Cooperative Oncology Group Performance Status score
^{a}The last non-missing ECOG score on or prior to date of Cilta-cel infusion is used. All patients met the inclusion criteria of ECOG score of 0 or 1 during screening.

### Baseline Disease Characteristics—Disease Burden

A possible causal relationship between the level of disease burden and the occurrence of the observed neurotoxicities was investigated. In the absence of publicly available criteria for defining high versus low disease burden, an exploratory analysis in which the categories of disease burden we defined as follows. A subject was categorized as having high tumor burden when any of the following parameters were met: (i) plasmacytosis in the bone marrow ≥80%; (ii) serum M-spike ≥5 g/dL; and (iii) serum free light chains ≥5000 mg/L. A subject was categorized as having low tumor burden when all of the following (as applicable to subject) parameters were met: (i) plasmacytosis <50%; (ii) serum M-spike <3 g/dL; and (iii) serum free light chains <3000 mg/L. Subjects who did not fit either criteria were considered to have intermediate disease burden.

When the criteria above were applied, high baseline disease burden appeared to be associated with higher CAR-T expansion and neurotoxicity (Table 6). Low baseline disease burden seemed associated with a lower incidence of neurotoxicity.

TABLE 6
Subject Baseline Disease Characteristics;
All Treated Analysis Set (Study 68284528MMY2001)

| | Movement and Neurocognitive | | |
|---|---|---|---|
| | Treatment-Emergent Adverse Events | | |
| | No | Yes | Total |
| | N = 92 | N = 5 | N = 97 |
| Baseline Tumor Burden Category | n (%) | n (%) | |
| High | 13 (14.1) | 3 (60.0) | 16 |
| Intermediate | 21 (22.8) | 1 (20.0) | 22 |
| Low | 58 (63.0) | 1 (20.0) | 59 |
| Baseline High Tumor Burden | | | |
| No | 79 (85.9) | 2 (40.0) | 81 |
| Yes | 13 (14.1) | 3 (60.0) | 16 |
| Baseline Type of Myeloma | | | |
| IgG | 55 (59.8) | 2 (40.0) | 57 |
| Non-IgG | 37 (40.2) | 3 (60.0) | 40 |
| Baseline Measurable Disease | | | |
| Type | | | |
| Serum only, Serum and Urine | 51 (55.4) | 4 (80.0) | 55 |
| Urine only, FLC, Not | 41 (44.6) | 1 (20.0)^{a} | 42 |
| evaluable | | | |
| Presence of Extramedullary | | | |
| Plasmacytoma at Baseline | | | |
| No | 80 (87.0) | 4 (80.0) | 84 |
| Yes | 12 (13.0) | 1 (20.0) | 13 |
| Presence of Bone-based | | | |
| Plasmacytoma at Baseline | | | |
| No | 87 (94.6) | 4 (80.0) | 91 |
| Yes | 5 (5.4) | 1 (20.0) | 6 |

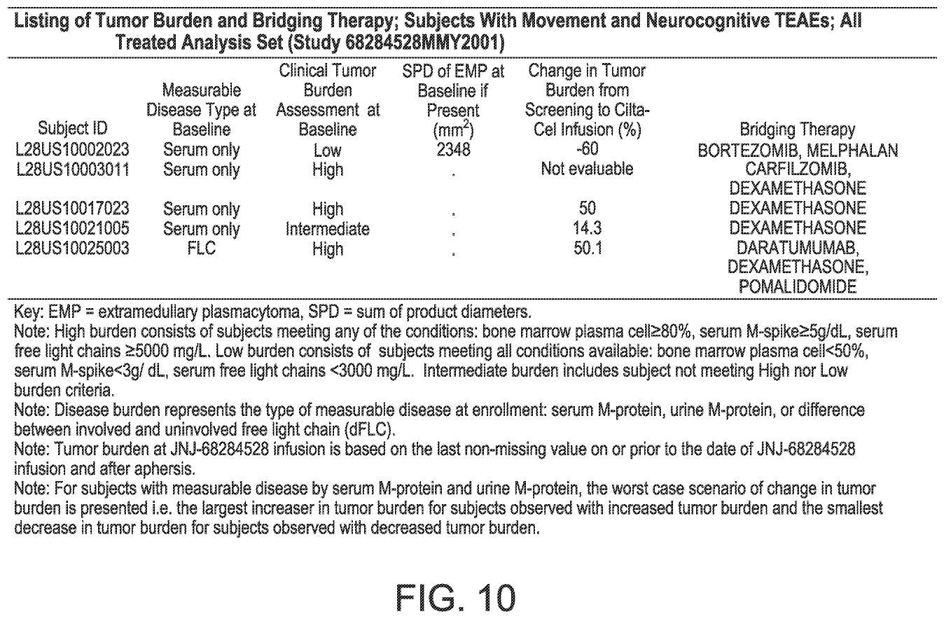

FLC = free light chain;
IgG = immunoglobulin G
^{a}Subject FLC-evaluable
Note:
High burden consists of subjects meeting any of the conditions: bone marrow plasma cell ≥80%, serum M-spike ≥5 g/dL, serum free light chains ≥5000 mg/L. Low burden consists of subjects meeting all conditions available: bone marrow plasma cell <50%, serum M-spike <3 g/dL, serum free light chains <3000 mg/L. Intermediate burden includes subject not meeting High nor Low burden criteria.
Cross reference: FIG. 10 and FIG. 12.

### Prior Treatment for Multiple Myeloma and Bridging Therapies for the Treatment of Multiple Myeloma

No relationship was identified between prior treatments and bridging therapies for Min and the occurrence of the observed movement/motor dysfunction and cognitive impairment TEAEs (Table 7). All 5 subjects received bridging therapy between apheresis and start of the conditioning regimen. After bridging therapy, 4 of the 5 subjects (800) experienced an increase in tumor burden (3 with increasing paraprotein, 1 with enlarging plasmacytoma despite improvement in paraprotein).

TABLE 7
Prior and Bridging Therapies for Multiple Myeloma;
All Treated Analysis Set (Study 68284528MMY2001)

| | Movement and Neurocognitive | | |
|---|---|---|---|
| | Treatment-Emergent | | |
| | Adverse Events | | |
| | No | Yes | Total |
| | N = 92 | N=5 | N = 97 |
| Number Lines of Prior | n (%) | n (%) | |
| Therapy Group | | | |
| 3-5 | 46 (50.0) | 3 (60.0) | 48 |
| >=6 | 46 (50.0) | 2 (40.0) | 49 |
| Prior Radiotherapy Including | | | |
| Brain Area | | | |
| No | 84 (91.3) | 5 (100.0) | 89 |
| Yes | 8 (8.7) | 0 (0.0) | 8 |
| Bridging Therapy Use | | | |
| No | 24 (26.1) | 0 (0.0) | 24 |
| Yes | 68 (73.9) | 5 (100.0) | 73 |
| Bridging Daratumumab Use | | | |
| No | 78 (84.8) | 4 (80.0) | 82 |
| Yes | 14 (15.2) | 1 (20.0) | 15 |
| Bridging Lenalidomide Use | | | |
| No | 86 (93.5) | 5 (100.0) | 91 |
| Yes | 6 (6.5) | 0 (0.0) | 6 |

Cross reference: FIG. 10

### Chimeric Antigen Receptor T-Cell Expansion and Chimeric Antigen Receptor T-Cell Persistence

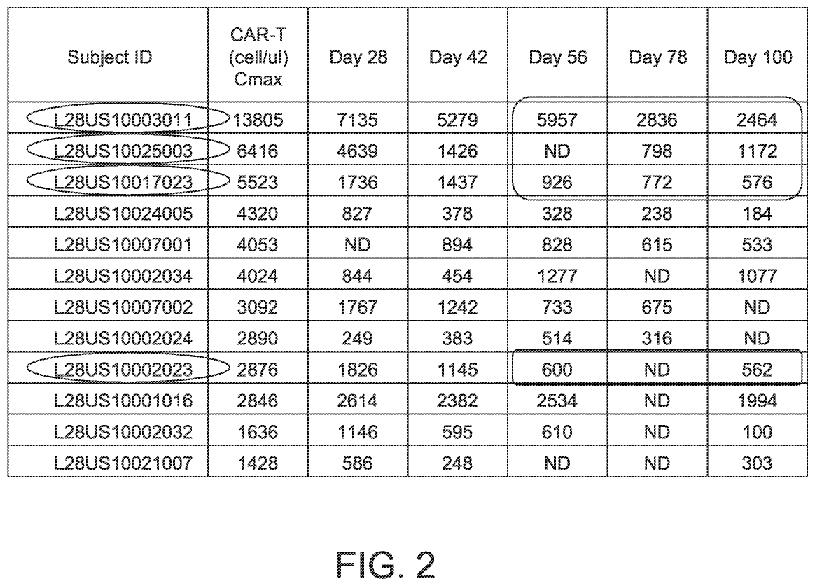

In Study 68284528MMY2001, CAR-T expansion and persistence was associated with a movement and neurocognitive TEAEs (Table 8), and subjects L28US10003011, L28US10025003, L28US10017023, and L28US10002023 were among the subjects with the highest peripheral blood CAR-T levels (FIG. 2) (Subject L28US10021005 was not among the subjects with a high cell expansion). Furthermore, the subjects with movement or neurocognitive TEAEs (Subjects L28US10002023, L28US10003011, L28US10017023, L28US10021005, and L28US10025003, (FIG. 12)) in particular had substantially longer persistence of peripheral blood CAR-T than subjects who did not develop these Other Neurotoxicity symptoms (FIG. 2). Cerebral spinal fluid (CSF) examinations for 2 subjects with movement or neurocognitive TEAEs (for whom CSF was available) showed that CAR-T cells represented the predominant fraction of the T cells which were primarily of effector memory phenotype.

These data suggest that a high level of CAR-T cell expansion and CAR-T cell persistence is associated with an increased risk of development of movement and neurocognitive TEAEs after resolution of CRS. It is possible that high levels of CAR-T cell expansion and CAR-T cell persistence contributed to the development of these Other Neurologic toxicities.

TABLE 8
CAR-T Therapy and Cell Expansion and Cell Persistence; All Treated
Analysis Set (Study 68284528MMY2001)

| | Movement and Neurocognitive | | |
|---|---|---|---|
| | Treatment-Emergent Adverse Events | | |
| | No | Yes | Total |
| | N = 92 | N = 5 | N = 97 |
| Total CAR-positive Viable | n (%) | n (%) | |
| T-cells Infused (×106 cells) | | | |
| <median value | 46 (50.0) | 2 (40.0) | 48 |
| >=median value | 46 (50.0) | 3 (60.0) | 49 |
| High Cell | | | |
| Expansion/Persistence^{a} | | | |
| No | 84 (91.3) | 1 (20.0) | 85 |
| Yes | 8 (8.7) | 4 (80.0) | 12 |

CAR = chimeric antigen receptor;
T-cells = T-lymphocytes
^{a}Subjects with Peripheral Blood CAR-T cells Cmax of >1000 cells/μL and CAR-T cells >300 cells/μL at Day 56

### Prior or Concurrent Cytokine Release Syndrome and Immune Effector Cell-Associated Neurotoxicity Syndrome

The correlation between prior or concurrent presence of CRS and ICANS and the development of movement and neurocognitive TEAEs was investigated (Table 9). All subjects with movement or neurocognitive TEAEs had ≥Grade 2 CRS whereas none of the subjects with <Grade 1 CRS reported this type of Other Neurotoxicity. Subjects with ICANS (any grade) were more prone to develop movement and neurocognitive TEAEs than subjects without ICANS (80.0% and 20.0%, respectively). Therefore, higher grade CRS (Grade 2 and above) and any grade ICANS appear to be associated with movement and neurocognitive TEAEs.

TABLE 9
Cytokine Release Syndrome and Immune Effector Cell-Associated
Neurotoxicity Syndrome;
All Treated Analysis Set (Study 68284528MMY2001)

| | Movement and | | |
|---|---|---|---|
| | Neurocognitive | | |
| | Treatment-Emergent | | |
| | Adverse Events | | |
| | No | Yes | Total |
| | N = 92 | N = 5 | N = 97 |
| Cytokine Release Syndrome | n (%) | n (%) | |
| (CRS) | | | |
| No | 5 (5.4) | 0 (0.0) | 5 |
| Yes | 87 (94.6) | 5 (100.0) | 92 |
| CRS Maximum Grade | | | |
| <2 | 54 (58.7) | 0 (0.0) | 54 |
| >=2 | 38 (41.3) | 5 (100.0) | 43 |
| ICANS | | | |
| No | 80 (87.0) | 1 (20.0) | 81 |
| Yes | 12 (13.0) | 4 (80.0) | 16 |

CRS = cytokine release syndrome;
ICANS = Immune Effector Cell-Associated Neurotoxicity Syndrome;
NTX = neurotoxicity

### Clinical Laboratory Values

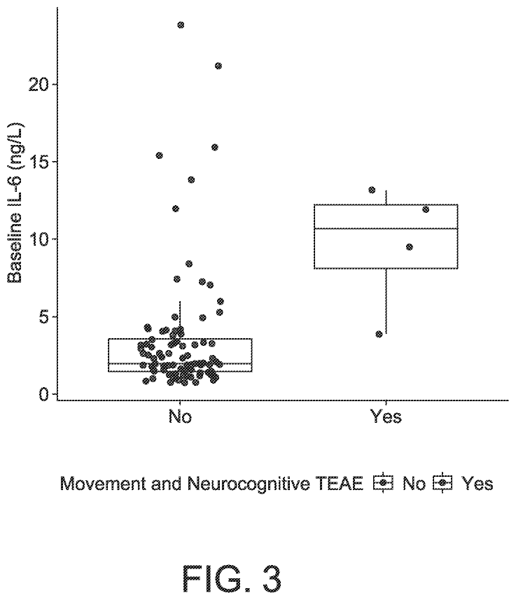

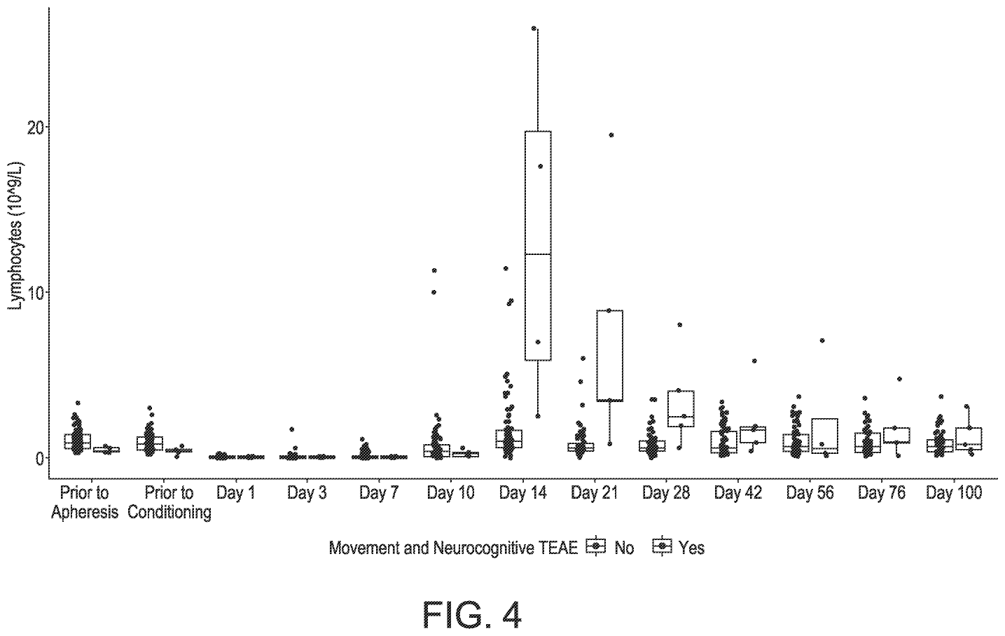

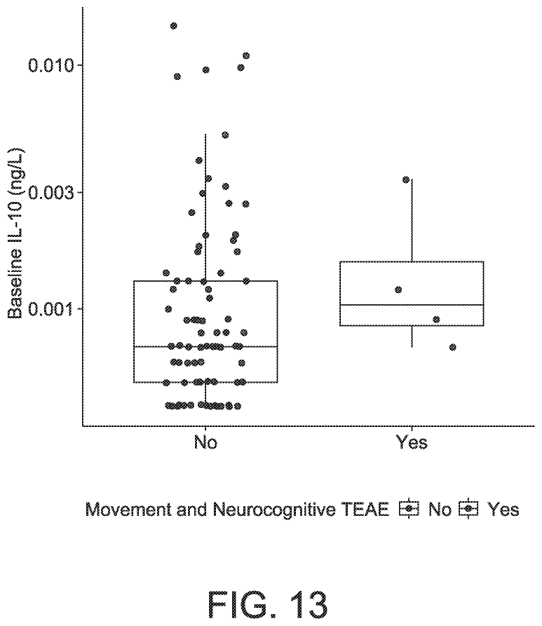

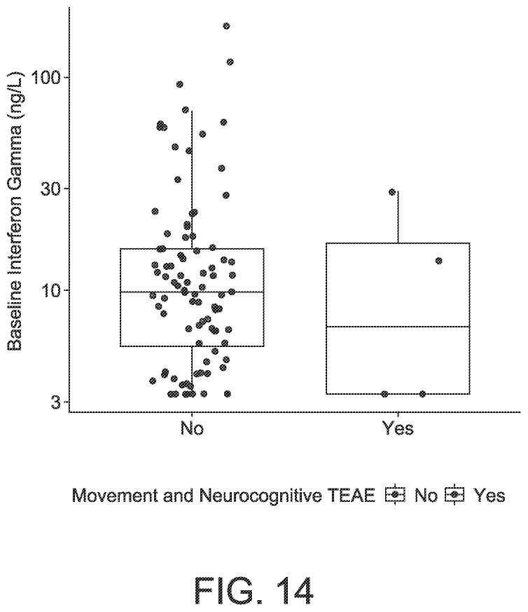

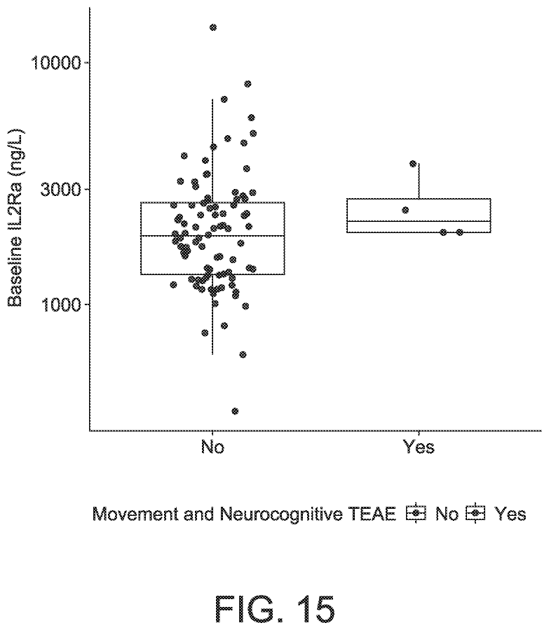

Early in the time course of cilta-cel infusion, clinical laboratory values associated with the risk and onset of movement and neurocognitive TEAEs include high baseline (i.e., prior to cilta-cel infusion) IL-6 (FIG. 3) and high lymphocyte counts on Study Days 14, 21, and 28 post cilta-cel infusion (FIG. 4). Data for additional baseline variables (IL-10, INF-γ, and IL2Ra) is provided in FIGS. 13-15.

### Biomarker Analyses—Peak Cytokine Levels

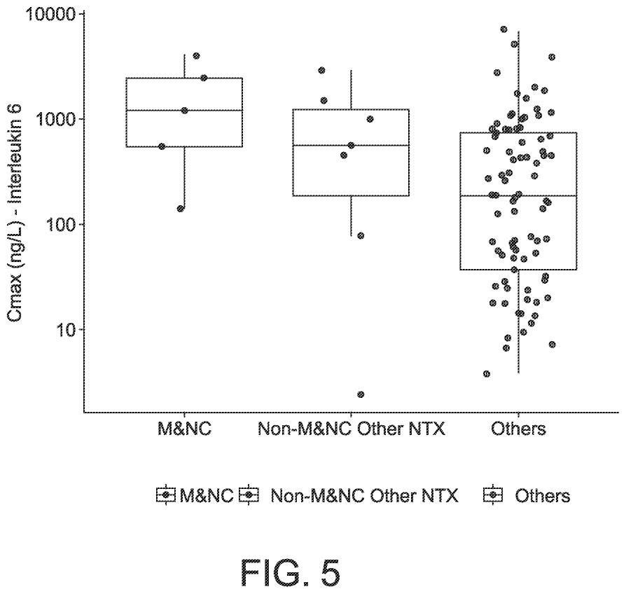

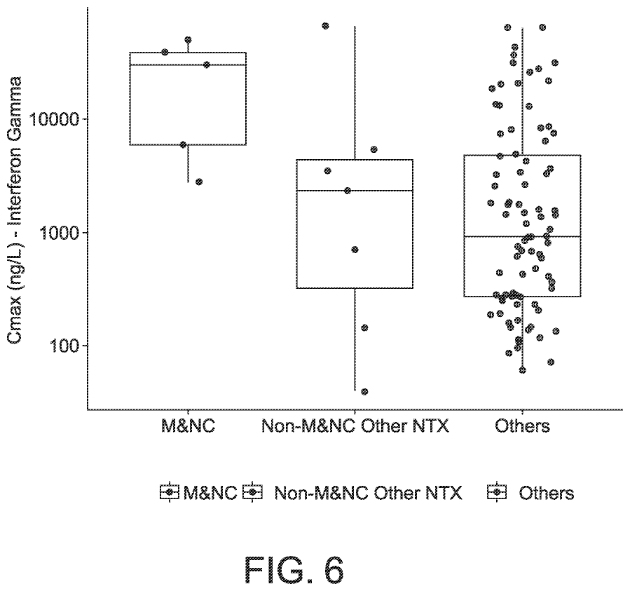

The median peak levels (Cₘₐₓ) of several proinflammatory cytokines in the peripheral blood, including IL-6 (FIG. 5) and INF-γ (FIG. 6), were elevated in subjects with movement and neurocognitive TEAEs as compared with subjects with Other Neurotoxicities or subjects who did not have a neurotoxic TEAE. Therefore, both biomarkers are considered to be associated with movement and neurocognitive TEAEs.

### Biomarker Analyses—T Cell Memory Phenotype at Apheresis

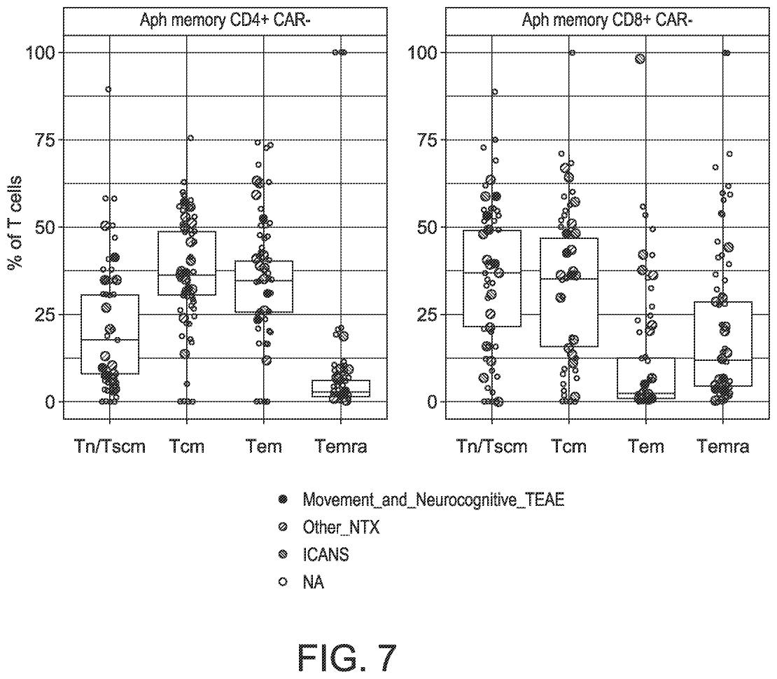

The memory phenotype of peripheral T cells was evaluated at the time of apheresis using standard flow cytometry methods. FIG. 7 shows the frequency distribution of CD4 and CD8 T cells of naïve/stem cell memory (Tn/Tscm), central memory (Tcm), effector memory (Tem) and effector memory re-expressing CD45RA (Temra) phenotype. The frequency of T cell memory subsets in subjects with Movement and Neurocognitive TEAEs overlapped with that of other subjects with Other neurotoxicity, ICANS or others.

### Neuropathology Findings at Autopsy

Of the 5 subjects in clinical study 68284528MMY2001 who experienced Other Neurotoxicities characterized by movement and neurocognitive TEAEs, 3 subjects died. The cause of death was neurotoxicity for 1 subject, lung abscess for 1 subject (autopsy was performed), and septic shock for the final subject (autopsy was performed). Neuropathology reports for the 2 subjects who had an autopsy performed showed focal gliosis and T cell infiltrate (CD8⁺>CD4⁺) in the basal ganglia. It is unknown if these T-lymphocytes are CAR-T⁺ cells. No abnormalities were reported for either subject in other brain regions that could potentially be associated with a movement TEAE (e.g., cerebellum, substantia nigra). Preservation of pigmentation in the substantia nigra was reported for both subjects.

### Immunohistochemical Evaluation of B-Cell Maturation Antigen Expression

To determine if movement and neurocognitive TEAEs could be related to target expression, B-cell migration antigen (BCMA) expression in the normal (non-diseased) human brain was evaluated. This investigation concluded that BCMA expression could not be detected in the normal (non-diseased) adult human brain.

An immunohistochemistry (IHC) assay using a commercial mAb (clone E6D7B) from Cell Signaling Technology, Inc. was developed for application to formalin-fixed paraffin-embedded (FFPE) brain samples. Two additional IHC assays employing a second commercial mAb (clone D6) from Santa Cruz Biotechnology, Inc. were developed at third-party molecular pathology laboratories also for application to FFPE samples. Both antibody clones (E6D7B and D6) sensitively and specifically detected BCMA in FFPE tissue and cell line controls (EDMS-RIM-367752, EDMS-RIM-367755, EDMS-RIM-387220).

Immunohistochemistry was performed internally on a total of 107 commercially sourced FFPE human brain samples spanning 63 individual donors and covering all regions of the brain (EDMS-RIM-387220). All samples included in the study were quality checked to confirm the location and suitability for IHC analysis. This internal assay used the E6D7B clone. Sporadic immunoreactivity was detected in the striatum and brainstem, and to a lesser extent in the thalamus, midbrain, hippocampus, and cerebellum. The immunoreactivity presented as fibrils and aggregates within neuronal cell bodies of the gray matter or as short thin threads along glial processes. This immunoreactivity was not reproduced when IHC was repeated using the D6 clone at third-party laboratories.

Due to the conflicting results seen with the two mAb clones, additional investigations were performed. Based on the results outlined below, it was determined that the immunoreactivity observed with the E6D7B clone represented nonspecific cross-reactivity and was not a reflection of true BCMA expression.

In situ hybridization using a BCMA-specific ribonucleic acid (RNA) probe from RNAscope (ACD Bio) was performed on 25 randomly selected brain samples previously stained with the E6D7B clone. B-cell maturation antigen RNA was not detected in the regions/neurons corresponding to E6D7B-mediated immunoreactivity (EDMS-RIM-387220).

Subcellular localization of neuronal immunoreactivity seen with the E6D7B clone is not consistent with our current understanding of BCMA expression biology. In BCMA-expressing plasma and MM cells, BCMA protein is detected on the cell membrane and within the Golgi apparatus (Gras 1995 et al., “BCMAp: An Integral Membrane Protein in the Golgi Apparatus of Human Mature B Lymphocytes,” Internat. Immunol. 7:1093-1106 (1995), which is hereby incorporated by reference in its entirety). No membranous immunoreactivity was detected in brain samples. Furthermore, confocal microscopy, using the E6D7B clone, found that immunoreactivity within neuronal cell bodies did not co-localize with Golgi-specific markers (EDMS-RIM-387220).

The immunoreactivity pattern seen with the E6D7B clone does not correlate with previously reported BCMA-expression data. A literature review and examination of publicly available BCMA-expression data was performed. Low levels of BCMA RNA can be detected in the striatum during fetal development, with levels decreasing through young adulthood. Beyond 30 years of age, BCMA-RNA expression is negligible (Brainspan.org, “Atlas of the Developing Human Brain,” available at https://www.brainspan.org. Accessed on 17 Mar. 2021 and GTExPortal. Broad Institute of MIT and Harvard. available at https://www.gtexportal/home. accessed on 17 Mar. 2021, which are hereby incorporated by reference in their entirety). No BCMA RNA or protein was detected in the normal adult cerebrum, cerebellum, or brainstem at any age (Brainspan.org, “Atlas of the Developing Human Brain,” available at https://www.brainspan.org. Accessed on 17 Mar. 2021; Bu et al., “Pre-Clinical Validation of B Cell Maturation Antigen (BCMA) as a Target for T Cell Immunotherapy of Multiple Myeloma,” Oncotarget 9(40):25764-25780 (2018); GTExPortal. Broad Institute of MIT and Harvard. available at https://www.gtexportal/home. accessed on 17 Mar. 2021; Carpenter et al., “B-Cell Maturation Antigen is a Promising Target for Adoptive T-Cell Therapy of Multiple Myeloma,” Clin. Cancer Res. 19(8):2048-2460 (2013); and Krumbholz et al., “BAFF is Produced by Astrocytes and Up-Regulated in Multiple Sclerosis Lesions and Primary Central Nervous System Lymphoma,” J. Exp. Med. 201(2):195-200 (2005), which are hereby incorporated by reference in their entirety). In the current RIC investigation, immunoreactivity was seen with the E6D7B clone across multiple brain areas and in the striatum of donors aged 39 years to 85 years of age).

Immunohistochemistry was performed on FFPE brain samples from 4 cynomolgus macaques using the E6D7B clone. No immunoreactivity was detected in the brain, although the antibody worked as an IHC reagent on cynomolgus FFPE tissue controls (EDMS-RIM-387220).

Immunohistochemistry-stained tissues were examined by an external neuropathologist. The neuropathologist confirmed that the IHC assays included the appropriate control samples; and, although both mAb clones exhibited similar performance on tissue and cell line controls, only the E6D7B clone exhibited immunoreactivity in the brain. The neuropathologist suggested that the immunoreactivity seen with the E6D7B clone was most likely nonspecific (EDMS-RIM-387220).

### Pharmacokinetics

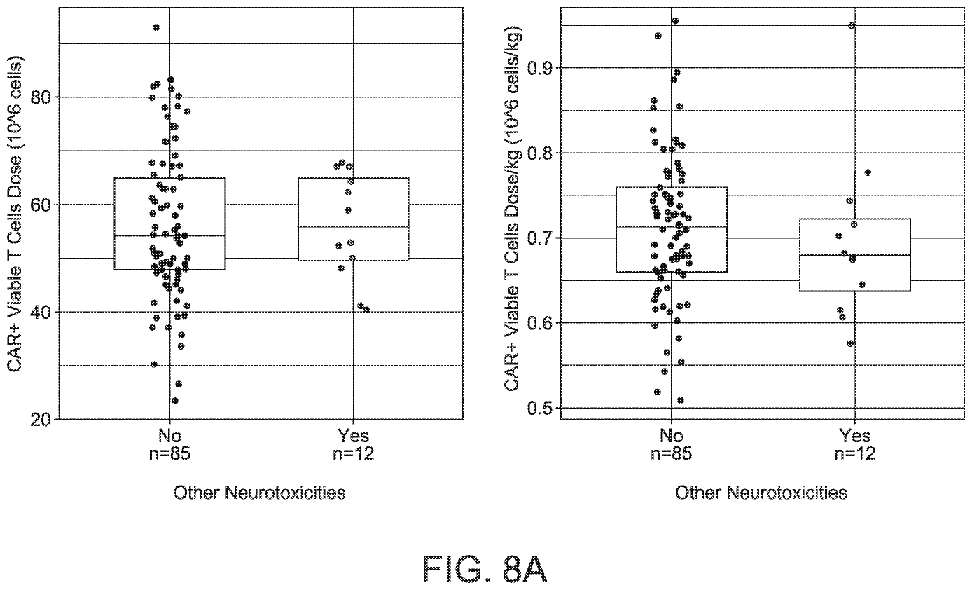

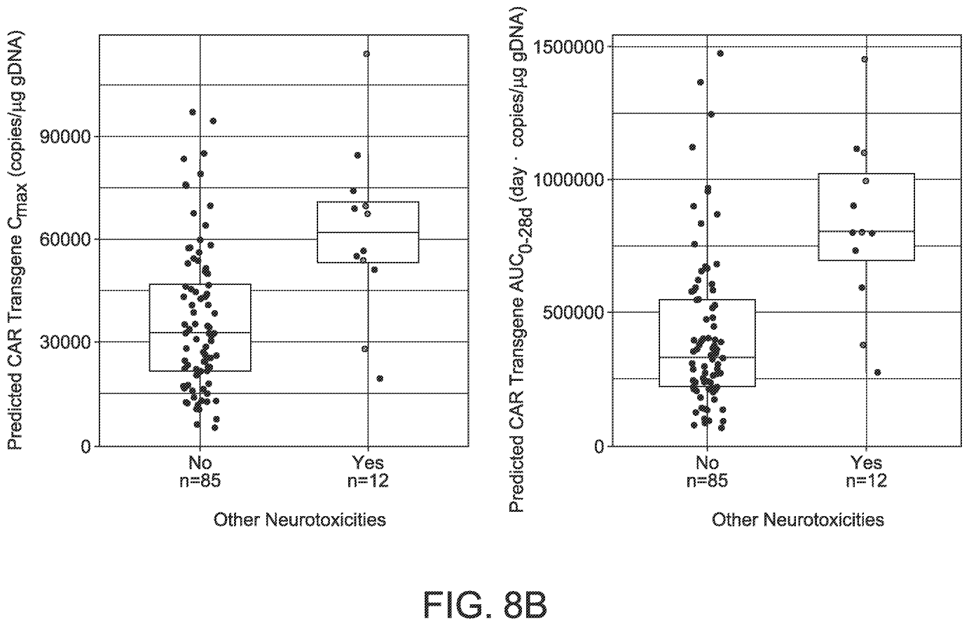

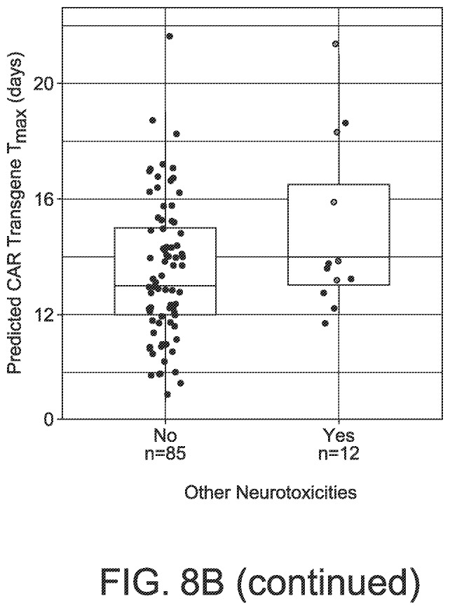

Exposure-response relationships for safety endpoints for Study 68284528MMY2001 are provided in FIGS. 8A-8B. No apparent trend with the infused cilta-cel total dose was observed for Other Neurotoxicities (including movement and neurocognitive TEAEs) (FIG. 8A). This is expected since only one target dose level (0.75 [range: 0.5-1.0]×10⁶ CAR-positive viable T-cells/kg) of cilta-cel was investigated in clinical study 68284528MMY2001.

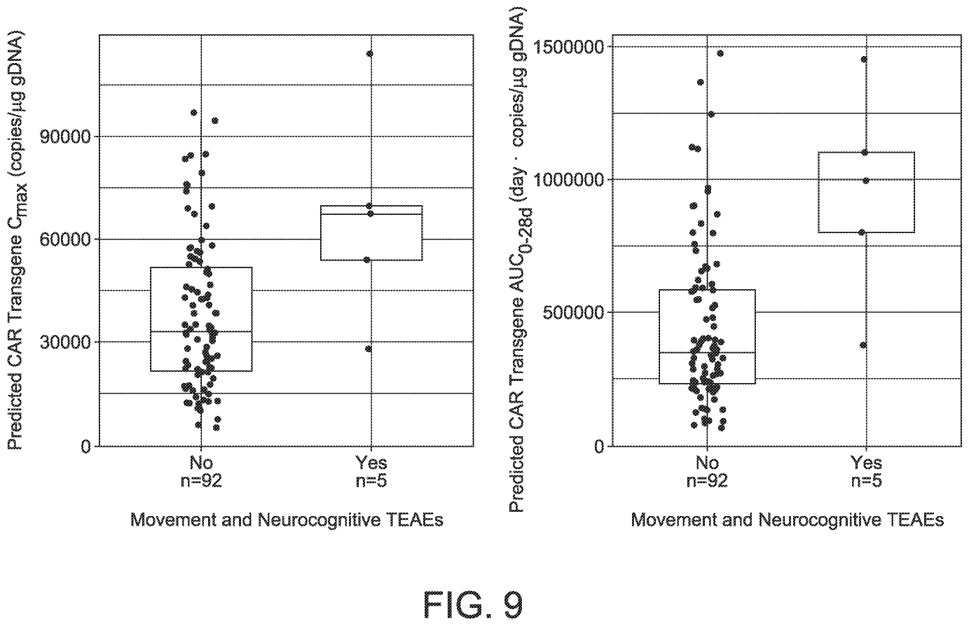

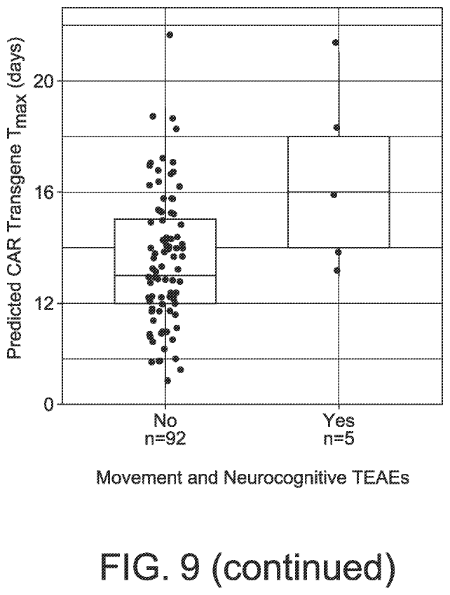

The median systemic CAR transgene levels (Cₘₐₓ and area under the time curve from the first dose to Day 28 (AUC_{0-28d})) in subjects with Other Neurotoxicities (including movement and neurocognitive TEAEs) or movement and neurocognitive TEAEs were generally higher than that in subjects without Other Neurotoxicities (including movement and neurocognitive TEAEs) (FIG. 8B) or movement and neurocognitive TEAEs (FIG. 9), respectively. However, the range of CAR transgene systemic exposure overlap among subjects without and with different grades of these TEAEs.

Similar to the Cₘₐₓ and AUC_{0-28d}, the ranges of time of maximum cilta-cel transgene expansion (Tₘₐₓ) in subjects without and with Other Neurotoxicities (including movement and neurocognitive TEAEs) were overlapping (FIG. 8B). Five subjects with movement and neurocognitive TEAEs appeared to have a delayed peak of expansion; however, no definitive conclusion could be drawn as the number of subjects is small (FIG. 9).

### Chemistry, Manufacturing, and Controls Assessment

Five subjects from the 68284528MMY2001 study experienced movement and neurocognitive TEAEs (subjects L28US10003011, L28US10025003, L28US1002023, L28US10017023, L2810021005). A thorough review of batch documentation was carried out for all 5 subject batches. Key drug product quality attributes have met the release specifications as outlined in Table 10 below, with the exception of batch 19HC0096, which was released per exceptional release procedure (reference IND 18080, S/N 0080 and 0085).

TABLE 10
Key Drug Product Quality Attributes (Study 68284528MMY2001)

| | | Date of | | |
|---|---|---|---|---|
| | Batch | Batch | Certificate of | Characterization |
| Subject ID | number | Release | Analysis Reference | Report Reference |
| L28US10003011 | 19GC0067 | 03 Jul. 2019 | Provided in S/N 0131 | Provided in S/N |
| | | | | 0131 |
| L28US10025003 | 19KC0177 | 10 Oct. 2019 | Provided in S/N 0131 | Appendix 5 |
| L28US10002023^{a} | 19HC0096 | 30 Aug. 2019 | Provided in S/N 0131 | Provided in S/N |
| | | | | 0131 |
| L28US10017023^{b} | 19JC0127 | 23 Sep. 2019 | Appendix 2 | Appendix 6 |
| L28US10021005^{b,c} | 19KC0165 | 03 Oct. 2019 | Appendix 3 | Appendix 7 |

^{a}Failure to meet dose specification (0.47 CAR-T viable cells × 10⁶/kg subject weight)
^{b}Cell clumps observed in drug product during visual inspection
^{c}Deviations occurred during processing and were deemed to have no impact on the final drug product. See text for details.

Manufacturing of Cilta-cel drug product in clinical study 68284528MMY2001 has been performed at Cincinnati Children's Hospital Medical Center (CCHMC), Janssen, Spring House, Pa. and Janssen, Raritan, N.J. The above 5 subject batches (Table 10) were all manufactured at the Raritan facility using vector batches LV-LICAR2SINV8008 (19GC0067 and 19HC0096) and LICAR2SINV8010 (19KC0177, 19JC0127, and 19KC0165). A total of 97 batches were manufactured for the 68284528MMY2001 study, with 68 batches manufactured at the Raritan site.

The 5 subject batches listed in Table 10 were investigated for any events or deviations that may have occurred during manufacturing. There were no significant events or deviations reported during the manufacture of these batches, with the exception of batch 19KC0165. In-process control testing for all 5 subject batches met acceptance criteria, and all release results also met acceptance criteria with the exception of the dose specification for batch 19HC0096. The release data is distributed across the manufacturing experience range. Cell clumping was observed in 2 batches of the drug product and are documented per the visual inspection Standard Operating Procedure. Cell clumping is not unexpected in CAR-T processes.

During processing of batch 19KC0165 on Day 10, the ambient room temperature dropped below the alarm limit of 55° F. due to a malfunction of the reheat system from water line that was inadvertently cut by a contractor working in the facility. The reheat system is responsible for the addition of heat to previously cooled air within the facility and is achieved by heating the hot water heating element in the air terminals. During 10-day processing, the cells are harvested and formulated in cryopreservation media that is stored at 2-8° C. (35.6 to 46.4° F.). The temperature excursion observed in the room is within the normal processing range temperature of ambient to 2° C. for this step. Additionally, a second deviation occurred during the control rate freezing (CRF) step for this batch, where a sensor fault error was observed upon placing the samples into the chamber. The CRF cycle was aborted and restarted and continued as expected. Both deviations were deemed to have no impact on the resulting drug product.

Process characterization data for T-cell expansion post-transduction, % CAR⁺CD4⁺, % CAR⁺CD8⁺, % CAR⁺/CD45⁺, % CAR⁺/CCR7⁺ for the 5 subject batches (Table 10) of cilta-cel were compared against data sets from across all 3 drug product manufacturing sites and were evaluated for any trends. Batch 19GC0067 showed T-cell expansion post-transduction and CD4⁺/CD8⁺ ratios post-selection at the upper end of the manufacturing range; however, this has been observed in other batches manufactured at CCHMC and Raritan, without correlation to a movement or neurocognitive event. The other 4 subject batches in this investigation display no observable trends, and the data are distributed across the manufacturing experience range for these attributes. The % CAR/CD45, CCR7 attributes show no trend in any of these 5 subject batches. Vector copy number for all 5 batches is below 0.5 copies per cell.

There is no assignable trend in the analytical in-process or release data for the 5 batches of cilta-cel drug product (Table 10) used to treat the 5 subjects with subsequent movement and neurocognitive TEAEs and occurrence of these adverse events. The process characterization attributes for all batches during manufacturing are continuously monitored in ongoing and planned clinical studies.

### SUMMARY

As more safety data with approved and emerging CAR-T therapies are generated, the definition of CAR-T related neurotoxicity will continue to evolve beyond ICANS to include Other Neurotoxicities. This document focused on Other Neurotoxicities categorized by a cluster of movement and neurocognitive adverse events in ongoing studies of cilta-cel. To date, possible factors associated with movement and neurocognitive adverse events include:
High tumor burden at baseline with high tumor burden defined as:
   Plasmacytosis in the bone marrow ≥80%, or
   Serum M-spike ≥5 g/dL, or
   Serum free light chains ≥5000 mg/L.
High baseline IL-6 levels;
High lymphocyte counts on Study Days 14, 21, and 28 (post cilta-cel infusion);
High peak levels (Cₘₐₓ) of IL-6 and INF-γ in peripheral blood;
Prior CRS (≥Grade 2) or ICANS (of any grade);
CAR-T cell expansion with a high level of cell expansion defined as a CAR-T cell Cₘₐₓ of >1,000 cells/μL; and
CAR-T cell persistence with a high level of cell persistence defined as CAR-T cells of >300 cells/μL at Study Day 56.

FIG. 11 summarizes the odds ratios and confidence intervals for each of these possible factors.

### Example 5—Mitigation Strategies to Reduce CAR-T Associated Neurotoxicity

Following the initial case of movement and neurocognitive TEAEs, an ad hoc Safety Management Team was convened, a notification to the United States (US) Food and Drug Administration (FDA) was sent, Dear Investigator Letters (DILs) were disseminated to active study sites (Studies 68274528MMY2001, 68284528MMY2002 and 68274528MMY2003), a Neurotoxicity Working Group was established, and routine Investigator teleconferences were conducted (Study 68274528MMY2001). In accordance with the DILs, Investigators were asked to alert the medical monitor to any neurotoxicities (including ICANS) post-cilta-cel infusion. Investigators were advised, as per ASTCT consensus criteria (included in the protocol), to treat cases of ICANS aggressively with steroids.

Given the cumulative cases of movement and neurocognitive TEAEs observed on clinical study 68274528MMY2001, the rapidly progressing severity (that included the inability to work or care for oneself following initial insidious subtle onset of symptoms), and the first fatal outcome, the immediate changes in the conduct of ongoing studies were implemented due to safety reasons that were subsequently followed by protocol and informed consent form amendments. The clinical summary and decision was communicated with the US FDA, and DILs were sent to all active sites (Studies 68274528MMY2001, 68284528MMY2002, 68274528MMY2003, and 68284528MMY3002). Based on emerging data, monitoring and mitigation strategies for movement and neurocognitive TEAEs were issued in program-wide protocol amendments.

The status of the studies conducted in the US (clinical studies 68284528MMY2001, 68284528MMY2003, and 68284528MMY3002) is summarized in Table 12.

TABLE 12
Overview of Ongoing Studies Conducted in the United States of America

| Study Number | Study Design | Objectives | Region | Status |
|---|---|---|---|---|
| 68284528MMY2001 | A Phase 1b-2, open- | Safety | US, Japan | Phase 1b |
| (CARTITUDE-1) | label, multicenter | (Phase 1b) | | and Phase |
| | study to evaluate the | ORR | | 2 |
| | safety and efficacy of | (Phase 2) | | enrollment |
| | Cilta-cel in adult | | | completed |
| | subjects with relapsed | | | |
| Study Number | Study Design | Objectives | Region | Status |
| | or refractory multiple | | | |
| | myeloma. | | | |
| | Phase 1b N = at least | | | |
| | 24 and up to | | | |
| | 50 subjects | | | |
| | Phase 2 N ≈ 60 | | | |
| | subjects | | | |
| | Phase 2 (Japan) N ≈ 8 | | | |
| | subjects | | | |
| 68284528MMY2003 | A Phase 2, | Safety and | US,EU | First |
| (CARTITUDE-2) | multicohort, open- | efficacy | | subject |
| | label, multicenter | | | dosed |
| | study. Four patient | | | February |
| | populations of unmet | | | 2020 |
| | medical need will be | | | |
| | studied³ | | | |
| | N ≈ 20 subjects in each | | | |
| | cohort | | | |
| 68284528MMY3002 | A Phase 3 | Safety and | Worldwide, | First |
| (CARTITUDE-4) | Randomized Study | efficacy | excluding | subject |
| | Comparing Cilta-cel, | | China | dosed July |
| | a Chimeric Antigen | | | 2020 |
| | Receptor T cell | | | |
| | (CAR-T) Therapy | | | |
| | Directed Against | | | |
| | BCMA vs | | | |
| | Pomalidomide, | | | |
| | Bortezomib and | | | |
| | Dexamethasone | | | |
| | (PVd) or | | | |
| | Daratumumab, | | | |
| | Pomalidomide and | | | |
| | Dexamethasone | | | |
| | (DPd) in Subjects | | | |
| | with Relapsed and | | | |
| | Lenalidomide- | | | |
| | Refractory Multiple | | | |
| | Myeloma | | | |
| | N = 400 (200 per | | | |
| | treatment arm) | | | |

The following mitigation steps were identified and implemented.
1. Dear Investigator Letters were sent to Investigators on the 68284528MMY2001, 68284528MMY2003, and 68284528MMY3002 studies.
2. The 68284528MMY2001, 68284528MMY2003, and 68284528MMY3002 protocols were amended to include a description of the key features of the pattern of movement and neurocognitive TEAEs and the potential for a severely disabling outcome.
3. Informed consent was updated to include the recent movement and neurocognitive TEAEs as well as provision to send the Sponsor a CSF sample if a lumbar puncture is performed. Study 68284528MMY2003 and Study 68284528MMY3002 informed consent forms were amended to include a provision for autopsy samples.
4. For subjects with a history of pertinent neurologic disease (e.g., stroke, encephalitis) both the 68284528MMY2003 and 68284528MMY3002 protocols were amended to recommend a baseline MRI of the brain and an EEG.
5. The 68284528MMY2003 protocol was amended to allow for bridging therapy (based on the Investigator's choice) in an effort to reduce tumor burden prior to CAR-T infusion. Previously, only agents that a subject was previously exposed to were permitted as part of bridging therapy. The 68284528MMY2001 protocol was amended to allow for bridging therapy in the case of re-treatment. The 68284528MMY3002 protocol was amended to allow additional cycles of bridging based on the subject's clinical status, again, in an effort to reduce tumor burden prior to CAR-T infusion.
6. For subjects with large baseline disease burden, particularly those with progressive disease despite bridging therapy, a risk benefit discussion should occur prior to CAR-T infusion as these subjects potentially have higher risk of developing severe neurotoxicity characterized by movement, neurocognitive, and personality change TEAEs.
7. The 68284528MMY2001, 68284528MMY2003, and 68284528MMY3002 protocols were amended to include a recommendation for extended use of prophylactic antimicrobials (up to 6 months or longer as per institutional guidelines) or consistent with post-ASCT consensus guidelines.
8. The 68284528MMY2001, 68284528MMY2003 and 68284528MMY3002 protocols were amended to incorporate the recommendation for early and aggressive steroid treatment of any grade ICANS in an effort to evaluate whether this intervention may decrease the risk of movement and neurocognitive TEAEs developing after CRS resolution per Investigator judgment.
9. Monitoring and reporting of neurologic events (new or exacerbation of any pre-existing ones) has been extended to one-year post-infusion (instead of 100 days) with instructions to notify the medical monitor if any movement or neurocognitive symptoms are observed.
10. The 68284528MMY2001, 68284528MMY2003 and 68284528MMY3002 protocols were amended to incorporate a routine handwriting assessment that was given to the subject pre-infusion and periodically after infusion throughout the course of the study in order to explore handwriting changes as a potential early indicator of Other Neurotoxicities. The novel handwriting tool was developed by Janssen and consists of a sentence log and grading criteria (Grade 1 and Grade 2) for 3 preferred terms (agraphia, micrographia, and dysgraphia). Sites are instructed to report these as TEAEs in the electronic data base and to immediately notify the medical monitor if any changes are observed. In case the subject develops a neurotoxicity serious adverse event (SAE) after resolution of CRS, a copy of the log should be added as part of the source documents. The handwriting assessment will be in addition to the current Immune Effector Cell-Associated Encephalopathy Assessment Tool (ICE).
11. A recommended workup has been added for subjects developing Other Neurotoxicity events including guidance to rule out specific viral infections (CSF analysis to rule out human herpes virus [HHV]-6, HHV-7, John Cunningham virus [JCV], herpes simplex virus [HSV]-1,2 as well as serology for HHV-6, HHV-7 and JCV), CSF flow cytometry to rule out leptomeningeal disease and paraneoplastic etiologies, serum thiamin levels (with consideration of supplementing), imaging (i.e., PET scan or MRI perfusion, EEG).
12. The 68284528MMY2001, 68284528MMY2003 and 68284528MMY3002 protocols were amended to include considerations for therapy directed at reduction or elimination of CAR-T in cases of movement and neurocognitive TEAEs not responding to other interventions in consultation with the Sponsor.
13. In addition to the mitigation steps listed above, a Data Monitoring Committee (DMC) was established for Study 68284528MMY2003 to review all subjects treated regardless of cohort.
14. For Cohort D of Study 68284528MMY2003, the following additional mitigation steps were put in place:
   a. Staggered dosing by at least 4 weeks for first 5 subjects
   b. Initiate the first 5 subjects without lenalidomide
   c. DMC to determine whether safe to initiate lenalidomide after data from the first 5 subjects are reviewed;
   d. First 5 subjects with initiation of lenalidomide (subjects 6 to 10) to be staggered by at least 4 weeks

In addition, the multiple evaluations have been undertaken to assess the possible predictive factors and the underlying pathology of movement and neurocognitive TEAEs (see Example 4); the results of which are summarized herein.

### Planned Monitoring

In addition to the long-term follow-up study (Study 68284528MMY4002) for continued monitoring of cilta-cel treated subjects for up to 15 years, an observational post-authorization safety study using a registry is planned with the purpose of additional characterization of identified risks, further evaluation of potential risks and missing information with special focus on long-term safety.

### Guidance to Physicians

Follow the monitoring and mitigation strategies for risks outlined in the Investigator Brochure (see, e.g., Table 13 below). In addition, to the minimize risk of movement and neurocognitive TEAEs for subjects in the ongoing cilta-cel clinical development program, monitoring and mitigation strategies were implemented including enhanced bridging therapy to reduce baseline tumor burden, early aggressive treatment of CRS and ICANS, handwriting assessments for early detection of neurotoxicity symptoms, and extended monitoring and reporting time for neurotoxicity beyond 100 days post-cilta-cel infusion. To date, other than the 5 subjects in clinical study 68284528MMY2001 and the 1 subject in clinical study 68284528MMY2003, as presented above, no further cases of Other Neurotoxicities characterized by movement and neurocognitive TEAEs have been reported for any study in the cilta-cel development program.

TABLE 13
Risks and Mitigation Strategies

| Identified Risk | Mitigation Strategies |
|---|---|
| Neurologic Toxicity | Monitor closely for neurologic AEs, including CAR-T cell-related |
| | neurotoxicity (e.g., ICANS) and raised intracranial pressure/cerebral |
| | edema; follow guidance for management in protocol. Participants |
| | should be advised to seek medical evaluation if they notice new onset |
| | of headache, convulsions, speech disorders, visual disorders, |
| | disturbances in consciousness, confusion and disorientation, and |
| | coordination, balance disorders, or mental status changes. Notify the |
| | sponsor if participant is experiencing any grade ICANS. At the first |
| | sign of neurotoxicity, neurology consultation and evaluation should be |
| | considered. The Immune effector Cell-associated Encephalopathy |
| | (ICE) Assessment Tool (ICE-Tool) should be performed at baseline |
| | and daily after the first symptoms of neurotoxicity are suspected and |
| | until resolution. Hospitalization is required for ≥Grade 2 CAR-T cell- |
| | related neurotoxicity (e.g, ICANS). Consider non-sedating, anti- |
| | seizure medicines (e.g, levetiracetam) for seizure prophylaxis for any |
| | Grade 2 or higher neurologic toxicities. |
| | Other cytokine-targeting therapies (for example, IL-1) may be used |
| | based on institutional practice, especially for cases of neurotoxicity |
| | which does not respond to tocilizumab or corticosteriods. Therapy |
| | directed at reduction or elimination of CAR-T cells, including |
| | chemotherapy, may be considered in consultation with the sponsor for |
| | participants who develop neurotoxicity that remains severe or life- |
| | threatening following prior therapies, including tocilizumab and |
| | corticosteroids. |
| | Other Neurotoxicities |
| | Monitor closely for other neurotoxicities with clinical presentation for |
| | the duration of the study after infusion with cilta-cel. Symptoms may |
| | have subtle onset, and include a cluster of movement and |
| | neurocognitive TEAEs, including: changes in movement (e.g., |
| | micrographia or changes in handwriting, tremors, bradykinesia, |
| | rigidity, shuffling gait, impaired balance and coordination, difficulty |
| | writing, difficulty performing activities of daily living like dressing or |
| | feeding oneself), cognitive impairments (e.g., memory loss or |
| | forgetfulness, disturbance in attention, mental slowness or fogginess, |
| | difficulty speaking or slurred speech, difficulty reading or |
| | understanding words), and personality changes (e.g, reduced facial |
| | expression, flat affect, reduced ability to express emotion, less |
| | communicative, disinterest in activities). If those neurologic or |
| | psychiatric symptoms are noted, contact the medical monitor and refer |
| | the participant immediately to a neurologist for a full evaluation. |
| | A cluster of movement and neurocognitive TEAEs were observed at a |
| | higher frequency in participants with high burden of disease and in |
| | participants experiencing higher grade CRS (Grade 2 and above) and |
| | any grade ICANS. This may be indicative that ≥Grade 2 CRS or any |
| | grade ICANS are early indicators for the development of other |
| | neurotoxicity after a period of recovery from CRS and/or ICANS. |
| | Therefore, ≥Grade 2 CRS or any grade ICANS may represent an |
| | opportunity for early intervention and more aggressive supportive care |
| | (including steroids) especially in patients treated with a high tumor |
| | burden, may mitigate the risk for developing late, other neurotoxicity. |
| | Infection and sepsis were seen concurrently in many of these patients. |
| | Mitigation strategies for other neurotoxicity include enhanced bridging |
| | therapy to reduce baseline tumor burden, early aggressive treatment of |
| | CRS and ICANS, handwriting assessments for early detection of |
| | neurotoxicity symptoms, and extended monitoring and reporting time |
| | for neurotoxicity for the duration of the study. |

ADL = activities of daily living;
CAR-T = chimeric antigen receptor T-cell;
CRS = cytokine release syndrome;
ICE = Immune effector Cell-associated Encephalopathy;
G-CSF = granulocyte colony-stimulating factor;
HLH/MAS = hemophagocytic lymphohistiocytosis/macrophage activation syndrome;
ICANS = Immune-Effector Cell-associated Neurotoxicity Syndrome;
TEAEs = treatment-emergent adverse events
^{a}Adverse event of special interest

If any neurologic or psychiatric symptoms are noted (see below), the medical monitor should be contacted, and the subject should be referred immediately to a neurologist for a full evaluation. Subjects should be monitored for neurotoxicity for the duration of the study after cilta-cel infusion. Particular attention should be paid to the appearance of any of the following.

Movement and neurocognitive TEAS, often with subtle onset:
movement impairments (e.g., micrographia or changes in handwriting, tremors, bradykinesia, rigidity, shuffling gait, impaired balance and coordination, difficulty writing, difficulty performing activities of daily living like dressing or feeding oneself);
cognitive impairments (e.g., memory loss or forgetfulness, disturbances in attention, mental slowness or fogginess, difficulty speaking or slurred speech, difficulty reading or understanding words);
personality change (e.g., reduced facial expression, flat affect, reduced ability to express emotions, less communicative, disinterest in activities).
   Early detection, workup, and intervention may be important to prevent neurologic toxicity from worsening. The following is a list of potential diagnostics that should be considered in subjects with new neurologic symptoms:
PET/computed tomography (CT) of the brain and/or brain MRI with perfusion and an EEG.
Lumbar puncture to rule out infection (in particular JCV, herpes zoster virus (HZV), HSV-1/2, HHV-6, HHV-7, Epstein-Barr virus (EBV), cytomegalovirus (CMV)).
Serologic testing for HHV-6 and HHV-7 by polymerase chain reaction for viremia.
CSF flow cytometry and cytology should be considered to rule out leptomeningeal disease.
CSF analysis should be considered to rule out paraneoplastic syndromes.
Thiamine level (consider empiric thiamine replacement while awaiting results).
   Therapy directed at the reduction or elimination of CAR-T, including chemotherapy, may be considered in consultation with the Sponsor for subjects who develop neurotoxicity that remains unresponsive to other interventions.

### CONCLUSION

The examples presented herein demonstrate the use of the following strategies used to mitigate and further understand the pathology of movement and neurocognitive adverse events: (i) baseline magnetic resonance imaging (MRI) of the brain and a baseline electroencephalogram (EEG) for subjects with a history of pertinent neurologic disease (e.g., stroke, encephalitis); (ii) bridging therapy to reduce tumor burden prior to administration of an immune effector cell therapy (e.g., cilta-cel infusion)¹; (iii) prophylactic use of antimicrobials for up to 6 months or longer (as per institutional guidelines or as consistent with post-autologous stem cell transplant [ASCT]consensus guidelines); (iv) early and aggressive steroid treatment for any grade of ICANS; (v) an extended monitoring and reporting period for neurologic adverse events to a duration beyond 100 days following the administration of an immune effector cell therapy; (vi) handwriting assessments at baseline (e.g., prior to cilta-cel infusion) and during treatment in order to explore handwriting changes as a potential early indicator of movement and neurocognitive adverse events; (vii) a virology workup to rule out infection, cerebral spinal fluid (CSF) flow cytometry to rule out leptomeningeal disease and paraneoplastic etiologies, serum thiamin levels (with consideration to supplement), and brain imaging (e.g., positron emission tomography (PET) scan or MRI perfusion, EEG); and (viii) therapies directed at the reduction or elimination of CAR-T cells in cases of movement and neurocognitive adverse events not responding to other interventions. ¹ For subjects with a high baseline disease burden despite bridging therapy, a risk/benefit discussion should occur as these subjects have a higher risk of developing severe movement, neurocognitive, personality change adverse events.

The mitigation strategies as described above were initiated for all ongoing cilta-cel studies (Urgent Safety Measure (USM)). Since the implementation of the mitigation strategies, the incidence of movement and neurocognitive adverse events has decreased from 5% to 1%. Movement and neurocognitive adverse events will continue to be monitored and exploratory evaluations will continue to be conducted into the possible predictive and contributing factors for these adverse events.

As more safety data with approved and emerging CAR-T therapies are generated, the definition of CAR-T related neurotoxicity will continue to evolve beyond ICANS to include other neurotoxicities categorized as movement and neurocognitive adverse events. Indeed, parkinsonism has been reported for ide-cel, another CAR-T therapy directed against the BCMA target for patients with MM. Movement and neurocognitive adverse events will continue to be monitored and exploratory evaluations will continue to be conducted into possible predictive factors for and the underlying the pathology of these adverse events.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of reducing neurotoxicity associated with chimeric antigen receptor (CAR) T cell therapy, said method comprising:
administering CAR-T cell therapy to a subject;
determining one or more of (i) the subject's tumor burden prior to said administering, (ii) the subject's IL-6 levels at the time of said administering, (iii) CAR-T cell expansion in said subject after said administering, (iv) CAR-T cell persistence in peripheral blood of said subject after said administering, (v) development of grade ≥2 cytokine release syndrome (CRS) in said subject after said administering, (vi) development of immune effector cell associated neurotoxicity syndrome (ICANS) in said subject after said administering, (vii) peak peripheral blood levels of IL-6 in said subject following said administering, (viii) peak peripheral blood levels of INF-γ in said subject after said administering; and (iv) lymphocyte counts in said subject after said administering; and
administering a mitigating therapeutic to said subject, based on said determining, to reduce neurotoxicity associated with CAR-T cell therapy.

2. The method of claim 1, wherein the subject has multiple myeloma.

3. The method of claim 1, wherein the CAR-T cell therapy is a B-cell maturation agent (BCMA) CAR-T cell therapy.

4. The method of claim 3, wherein the BCMA CAR-T cell therapy is ciltacabtagene autoleucel (cilta-cel).

5. The method of claim 1, wherein the neurotoxicity associated with the CAR-T cell therapy comprises one or more movement and motor dysfunction adverse events, cognitive impairment adverse events, personality change adverse events, or any combination thereof.

6. The method of claim 1, wherein the neurotoxicity associated with the CAR-T cell therapy is not ICANS

7. The method of claim 1, wherein said determining is repeated periodically.

8. The method of claim 2, wherein determining the subject's tumor burden comprises:
measuring the subject's bone marrow plasmacytosis, serum M protein levels, serum free light chain levels, or a combination thereof.

9. The method of claim 8, wherein when said subject has a tumor burden characterized by bone marrow plasmacytosis of ≥80%, serum M protein levels of ≥5 g/dL, or serum free light chain levels of ≥5000 mg/L based on said determining, said mitigating therapeutic comprises a bridging therapy, said method further comprising:
administering the bridging therapy prior to administering the CAR-T cell therapy.

10. The method of claim 9, wherein said bridging therapy comprises a chemotherapeutic, an immunomodulatory agent, a proteasome inhibitor, or any combination thereof.

11. The method of claim 10, wherein the chemotherapeutic is an alkylating agent or a topoisomerase inhibitor.

12. The method of claim 10, wherein the immunomodulatory agent comprises a CD38 inhibitor.

13. The method of claim 12, wherein the CD38 inhibitor is daratumamab or isatuximab.

14. The method of claim 10, wherein the immunomodulatory agent is selected from lenalidomide, pomalidomide, thalidomide, and combinations thereof.

15. The method of claim 10, wherein the proteasome inhibitor is selected from bortezomib, carfilzomib, ixazomib, and combinations thereof.

16. The method of claim 1, wherein if the subject's IL-6 blood levels at the time of said administering are above the upper limit of normal, said mitigating therapeutic comprises an IL-6 inhibitor, said method further comprising:
administering the IL-6 inhibitor prior to administering the CAR-T cell therapy.

17. The method of claim 1, wherein if the subject's peak peripheral blood IL-6 levels after said administering are above the upper limit of normal, said mitigating therapeutic comprises an IL-6 inhibitor.

18. The method of claim 16 wherein the IL-6 inhibitor is tocilizimab.

18. The method of claim 17 wherein the IL-6 inhibitor is tocilizimab.

19. The method of claim 1, wherein if the subject's CAR-T cell expansion, based on said determining, is >1000 cells/μL after said administering, the mitigating therapeutic comprises a chemotherapeutic agent suitable for reducing the number of CAR-T cells in said subject.

20. The method of claim 19, wherein the chemotherapeutic agent is an alkylating agent or a topoisomerase inhibitor.

21. The method of claim 1, wherein if the subject's persisting CAR-T cell concentration in peripheral blood, based on said determining, is >300 cells/μL after said administering, the mitigating therapeutic comprises a chemotherapeutic agent suitable for reducing the number of CAR-T cells in said subject.

22. The method of claim 21, wherein the chemotherapeutic agent is an alkylating agent or a topoisomerase inhibitor.

23. The method of claim 1, wherein if said subject has grade ≥2 CRS or any grade of ICANS based on said determining, the mitigating therapeutic comprises an anti-inflammatory agent suitable for treating said CRS or ICANS.

24. The method of claim 23, wherein said anti-inflammatory agent is an IL-6 inhibitor.

25. The method of claim 24, wherein the IL-6 inhibitor is tocilizumab.

26. The method of claim 23, wherein said anti-inflammatory agent is a steroid.

27. The method of claim 26, wherein the steroid is selected from dexamethasone, prednisone, methylprednisolone, and combinations thereof.

28. The method of claim 1 further comprising:
monitoring the subject for symptoms of agraphia, micrographia, dysgraphia, or any combination thereof after administering the CAR-T cell therapy.

29. The method of claim 28, wherein if said subject, based on said monitoring, exhibits symptoms of agraphia, micrographia, or dysgraphia after said administering, said method further comprises:
(i) assessing a cerebral spinal fluid sample from the subject for the presence of infection, leptomeningeal disease, parananeoplastic syndrome, or a combination thereof,
(ii) determining the subject's serum levels of human herpes virus (HHV)-6, HHV-7, or both;
(iii) measuring the subject's serum thiamine levels;
(iv) imaging the subject's brain via positron emission tomography or magnetic resonance imaging;
(v) performing an electroencephalogram (EEG); or
(vi) any combination of (i), (ii), (iii), (iv) and (v).

30. A method for treating multiple myeloma in a subject with chimeric antigen receptor (CAR) T cell therapy while reducing neurotoxicity associated with said therapy, said method comprising:
administering CAR-T cell therapy to the subject having multiple myeloma, wherein said subject has a tumor burden characterized by bone marrow plasmacytosis of <80%, serum M Protein levels of <5 g/dL, and serum free light chain levels of <5000 mg/L.

31. The method of claim 30, wherein said subject has a tumor burden characterized by bone marrow plasmacytosis of <50%, serum M Protein levels of <3 g/dL, and serum free light chain levels of <3000 mg/L.

32. The method of claim 30 further comprising:
administering a bridging therapy to the subject to achieve a tumor burden characterized by bone marrow plasmacytosis of <80%, serum M Protein levels of <5 g/dL, and serum free light chain levels of <5000 mg/L prior to administering the CAR-T cell therapy.

33. The method of claim 32, wherein said bridging therapy comprises a chemotherapeutic, an immunomodulatory agent, a proteasome inhibitor or a combination thereof.

34. The method of claim 33, wherein the chemotherapeutic is an alkylating agent or a topoisomerase inhibitor.

35. The method of claim 33, wherein the immunomodulatory agent comprises a CD38 inhibitor.

36. The method of claim 35, wherein the CD38 inhibitor is daratumamab.

37. The method of claim 33, wherein the immunomodulatory agent is selected from lenalidomide, pomalidomide, thalidomide, and combinations thereof.

38. The method of claim 33, wherein the proteasome inhibitor is selected from bortezomib, carfilzomib, ixazomib, and combinations thereof.

39. A method for treating multiple myeloma in a subject with chimeric antigen receptor (CAR) T cell therapy while reducing neurotoxicity associated with said therapy, said method comprising:
administering the CAR-T cell therapy to said subject having multiple myeloma and an IL-6 serum level that is within normal reference range of 0-2 pg/mL.

40. The method of claim 39 further comprising:
administering, to the subject, an IL-6 inhibitor to achieve the normal reference range of IL-6 in the subject prior to administering the CAR-T cell therapy.

41. A method of reducing neurotoxicity in a subject receiving chimeric antigen receptor (CAR) T cell therapy for the treatment of multiple myeloma, said method comprising:
administering to the subject having received CAR-T cell therapy and having symptoms of CAR-T cell therapy associated cytokine release syndrome (CRS) or immune effector cell associated neurotoxicity syndrome (ICANS), an anti-inflammatory agent in an amount effective to reduce neurotoxicity in said subject.

42. The method of claim 41, wherein said anti-inflammatory is an IL-6 inhibitor.

43. The method of claim 42, wherein the IL-6 inhibitor is tocilizumab.

44. The method of claim 42, wherein said anti-inflammatory is a steroid.

45. The method of claim 44, wherein the steroid is selected from dexamethasone and methylprednisolone.

46. The method of claim 41, wherein the CRS is grade ≥2.

47. The method of claim 41, wherein the ICANS is grade ≥1.

48. A method of reducing neurotoxicity in a subject receiving chimeric antigen receptor (CAR) T cell therapy for the treatment of multiple myeloma, said method comprising:
administering, to the subject having received CAR-T cell therapy and having a CAR-T cell maximum plasma concentration (Cmax) of >1,000 cells/μL and/or a persisting CAR-T cell concentration in peripheral blood of >300 cells/μL after said CAR-T cell administration, a chemotherapeutic to reduce CAR-T cell therapy associated neurotoxicity.

49. A method of reducing neurotoxicity in a subject receiving chimeric antigen receptor (CAR) T cell therapy for the treatment of multiple myeloma, said method comprising:
administering, to the subject having received CAR-T cell therapy and having a peak peripheral blood IL-6 level that is above the upper limit of normal after CAR-T cell administration, an IL-6 inhibitor to reduce CAR-T cell therapy associated neurotoxicity.

50. The method of claim 30, wherein the CAR-T cell therapy is a B-cell maturation agent (BCMA) CAR-T cell therapy.

51. The method of claim 50, wherein the BCMA CAR-T cell therapy is ciltacabtagene autoleucel (cilta-cel).

52. The method of claim 29, wherein the neurotoxicity associated with the CAR-T cell therapy comprises movement adverse events, neurocognitive adverse events, personality change adverse events, or any combination thereof.

53. The method of claim 29, wherein the neurotoxicity associated with the CAR-T cell therapy is not ICANS.
